(19) United States
(12) Patent Application Publication
(10) Pub. No.: US 2016/0264617 A1
(43) Pub. Date: Sep. 15, 2016

(54) Integrated Approach to the Isolation and Purification of Antibodies

(71) Applicant: AbbVie Inc., North Chicago, IL (US)

(72) Inventors: Diane D. Dong, Shrewsbury, MA (US); Stephen M. Lu, Worcester, MA (US); Natarajan Ramasubramanyan, Westborough, MA (US); Wen-Chung Lim, Worcester, MA (US)

(21) Appl. No.: 14/883,195

(22) Filed: Oct. 14, 2015

Related U.S. Application Data
(63) Continuation of application No. 13/420,438, filed on Mar. 14, 2012, now abandoned.
(60) Provisional application No. 61/452,968, filed on Mar. 15, 2011.

(51) Int. Cl. C07K 1/18; B01D 15/42; C07K 1/36; B01D 15/36; C07K 16/18; C07K 1/16

(52) U.S. Cl. CPC C07K 1/18; C07K 16/18; C07K 1/165; C07K 1/36; B01D 15/363; B01D 15/426; C07K 2317/10

(57) ABSTRACT

Disclosed herein is an integrated approach to purification process development and execution, including processes comprising particular capture and fine purification steps; processes that employ of a minimal number of buffer systems, and processes that make use of minimally-corrosive buffer systems, as well as combinations thereof.

### CROSS-REFERENCE TO RELATED APPLICATIONS

This application claims priority to U.S. Provisional Application Ser. No. 61/452,968 filed Mar. 15, 2011, the disclosure of which is hereby incorporated by reference in its entirety.

### BACKGROUND OF THE INVENTION

Purification processes for pharmaceutical grade monoclonal antibodies produced by mammalian cell culture typically involve four basic steps. These steps include (1) harvest/clarification—separation of host cells from the cell culture broth; (2) capture—separation of antibody from the majority of components in the clarified harvest; (3) fine purification—separation or reduction of the antibody from residual host cell contaminants, other impurities and aggregates/product related substances; and (4) formulation—placing the antibody into an appropriate carrier/excipient(s) for maximum stability and shelf life.

However, merely practicing these steps does not necessarily result in antibody compositions of sufficient purity for use in pharmaceutical contexts. For example, in certain situations, the inclusion of multiple independent capture and/or fine purification separations is required. Additionally, the presence of corrosive ingredients in many of the buffers traditionally employed in commercial antibody production and the conventional strategy of using distinct buffering systems for individual separations can lead to increased costs, longer process development times, and extended purification run times. Thus, there is a present need for improved methods of purifying antibodies of interest for pharmaceutical use. The present invention addresses this need.

### SUMMARY OF THE INVENTION

The present invention is directed to the use of an integrated approach to purification process development and manufacture. Such an integrated approach allows, in certain embodiments, for a single platform purification process or system to be deployed for the purification of distinct antibodies and, in certain embodiments, for the realization of efficiencies by using limited numbers of buffer components and/or minimally corrosive, chloride free buffers.

In certain embodiments, the integrated approach concerns the development of processes comprising particular capture and fine purification steps. For example, in certain embodiments, the methods described herein can employ a single capture separation, such as a Protein A-based separation, followed by a single fine purification separation, such as a mixed mode separation. However, in certain embodiments, the fine purification step can include one, two, three or more individual separations, such as, but not limited to, a mixed mode separation followed by a anion or cation exchange membrane-based separation.

Another aspect of the invention, the integrated approach to purification process development concerns the use of a minimum number of buffer systems during the course of the process. For example, in certain embodiments, a single buffer system is employed throughout the capture and fine purification steps. In particular embodiments the buffer system will consist essentially of water and two other ionic components, namely an anionic component and a cationic component, with the two ionic components being mixed in different combinations and concentrations to create buffers suitable for the needs of any particular purification process.

In a further aspect of the invention, the integrated approach to purification process development concerns the use of a minimally-corrosive buffer system. Certain buffer systems employ salts, such as chloride salts, that can have corrosive impact on commercial antibody production and purification equipment. In certain embodiments, the present invention relates to buffer systems that employ minimally-corrosive, chloride free buffer systems, such as, but not limited to, those that employ either tris or trolamine paired with either acetate or citrate.

In certain embodiments the present invention relates to purification processes where, one, two or all three aspects of the integrated approach to purification process development are employed. For example, in certain embodiments, a purification process of the present invention will comprise a single capture separation and single fine purification separation, where both separations employ the same buffer system. Further, in certain of such embodiments, that single buffer system will be a minimally-corrosive, chloride free buffer system.

In certain embodiments, the present invention relates to methods for producing a host cell protein-reduced antibody preparation from a sample mixture comprising an antibody and at least one host cell protein, where the method comprises: (a) contacting the sample mixture with a loading buffer and contacting the loading buffer and sample mixture to a capture separation chromatographic support under conditions where the antibody is retained on the chromatographic support; (b) washing the capture separation chromatographic support with a wash buffer to remove the sample mixture components that are not retained on the capture separation chromatographic support; and (c) contacting the capture separation chromatographic support with an elution buffer to thereby produce a capture separation eluate; where the loading, wash, and elution buffers consist of water and essentially the same anion and cation components; and where the capture separation eluate comprises a host cell protein-reduced antibody preparation. In certain of such embodiments, the anion and cation components are selected from the group consisting of Tris and Citrate, Tris and Acetate; Trolamine and Citrate; and Trolamine and Acetate.

In certain embodiments, the methods of the present invention comprise: (a) contacting a sample mixture comprising an antibody and at least one host cell protein with a loading buffer and contacting the loading buffer and sample mixture to a capture separation chromatographic support under conditions where the antibody is retained on the chromatographic support; (b) washing the capture separation chromatographic support with a wash buffer to remove the sample mixture components that are not retained on the capture separation chromatographic support; (c) contacting the capture separation chromatographic support with an elution buffer to thereby produce a capture separation eluate; (d) contacting the capture separation eluate to a loading buffer and contacting the capture separation eluate and loading buffer mixture to a fine purification separation chromatographic support capable of further reducing the host cell protein content of the capture separation eluate; (e) washing the fine purification chromatographic support with a wash buffer to remove the capture separation eluate components that are not retained on the fine purification chromatographic support; and (f) contacting the fine purification chromatographic support with an elution buffer to thereby produce a fine purification separation eluate; wherein the capture separation and fine purification separation load, wash, and elution buffers consist of water and essentially the same anion and cation components selected from the group consisting of Tris and Citrate, Tris and Acetate; Trolamine and Citrate; and Trolamine and Acetate

In certain embodiments, the present invention relates to methods for developing an integrated purification protocol for producing host cell-reduced preparations from two sample mixtures where each sample mixture comprises a distinct antibody and at least one host cell protein: (a) selecting a capture separation chromatographic support capable of retaining the distinct antibodies of the sample mixtures; and (b) selecting load, wash, and elution buffers for, respectively, loading, washing, and production of a capture separation eluate; wherein the loading, wash, and elution buffers consist of water and essentially the same anion and cation components; and wherein the capture separation eluate comprises the host cell protein-reduced antibody preparation. In certain of such embodiments, the anion and cation components are selected from the group consisting of Tris and Citrate, Tris and Acetate; Trolamine and Citrate; and Trolamine and Acetate.

In certain embodiments, the present invention relates to methods for developing an integrated purification protocol for producing host cell-reduced preparations from two sample mixtures where each sample mixture comprises a distinct antibody and at least one host cell protein: (a) selecting a capture separation chromatographic support capable of retaining the distinct antibodies of the sample mixtures; (b) selecting load, wash, and elution buffers for, respectively, loading, washing, and production of a capture separation eluate; (c) selecting a fine purification separation chromatographic support capable of further reducing the host cell protein content of the capture separation eluate; and (d) selecting load, wash, and elution buffers for, respectively, loading, washing, and production of a fine purification separation eluate; wherein the capture separation and fine purification separation buffers consist of water and essentially the same anion and cation components selected from the group consisting of Tris and Citrate, Tris and Acetate; Trolamine and Citrate; and Trolamine and Acetate.

### BRIEF DESCRIPTIONS OF THE DRAWINGS

FIG. 1 illustrates the difference between a traditional mAb purification process that utilizes multiple buffers to a non-limiting example of an integrated purification platform process utilizing a simplified two component buffer system.

FIG. 2 depicts the impact certain buffer systems have on Protein A resins. Panel A (left panel) shows the impact of acetic, phosphoric and citric acid systems on elution pH transitions (thick lines) during the product elution (UV signal shown in thin lines). Panel B (right panel) shows the impact of selected buffer systems on HCP reduction (as measured in the eluate pool) for selected Protein A resins. Antibody A was used in this study.

FIG. 3 depicts a summary of four representative integrated purification processes for antibody A as compared to a traditional process in terms of process step yield (panel A) and impurity removal (host cell proteins—panel B, aggregates—panel C, and leached Protein A—panel D). For each step, the processes described are presented in the following order: (i) mAbSelect Sure—F0HC depth filter—Capto Adhere—Chromasorb Q—Virosart; (ii) Prosep Ultra Plus—F0HC depth filter—Capto Adhere—Chromasorb Q—Virosart; (iii) mAbSelect Sure—F0HC depth filter—Nuvia S—Chromasorb Q—Virosart; (iv) Prosep Ultra Plus—F0HC depth filter—Nuvia S—Chromasorb Q—Virosart; (v) mAbSelect Sure—F0HC depth filter—Q Sepharose—Phenyl HP Sepharose—Virosart.

FIG. 4 depicts HCP reductions across post-low pH inactivation depth filtration for two molecules and selected depth filters. The resultant HCP content is shown in bars and the throughput is shown as points. The effect of pH inactivation alone is shown with the control 0.2 μm filter.

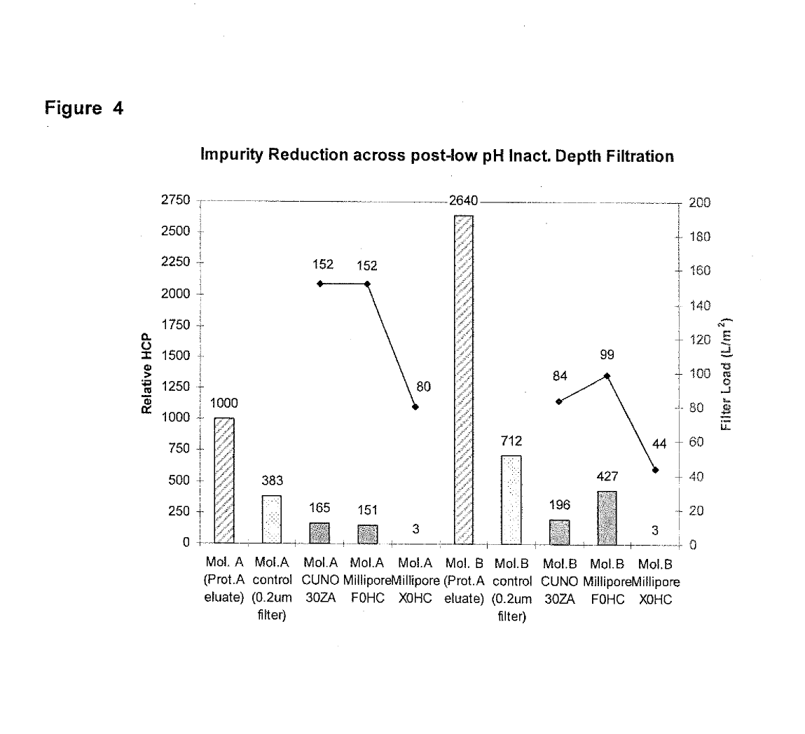

FIG. 5 depicts the impact of selected minimally corrosive two component buffer systems on depth filter performance in terms of throughput (left panel) and host cell protein breakthrough profile (right panel) for antibody A.

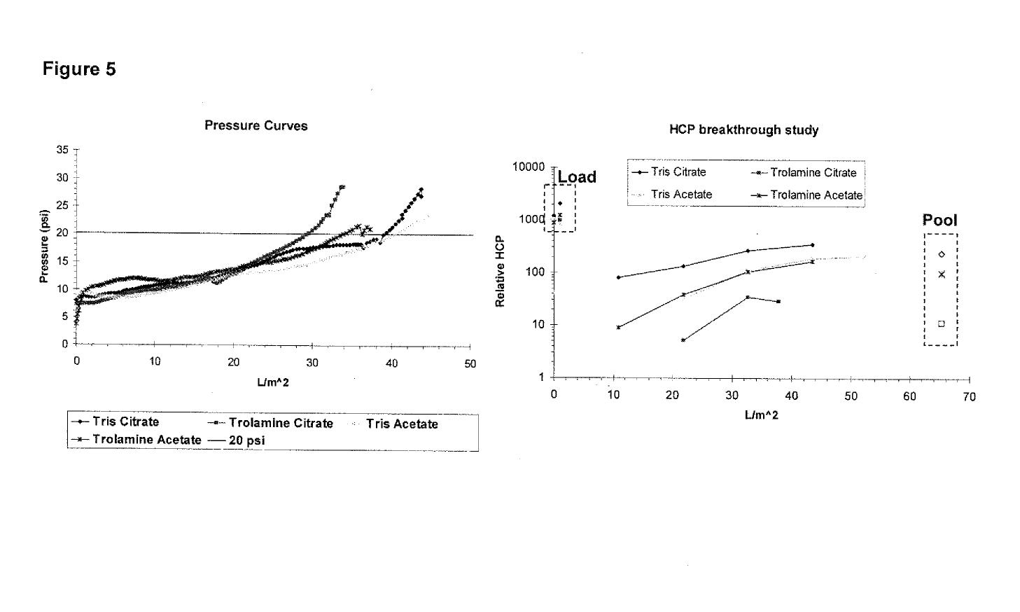

FIG. 6 depicts HCP breakthrough profiles of Protein A eluate loaded onto a Chromasorb Q membrane in selected minimally corrosive two component buffer systems) for antibody A.

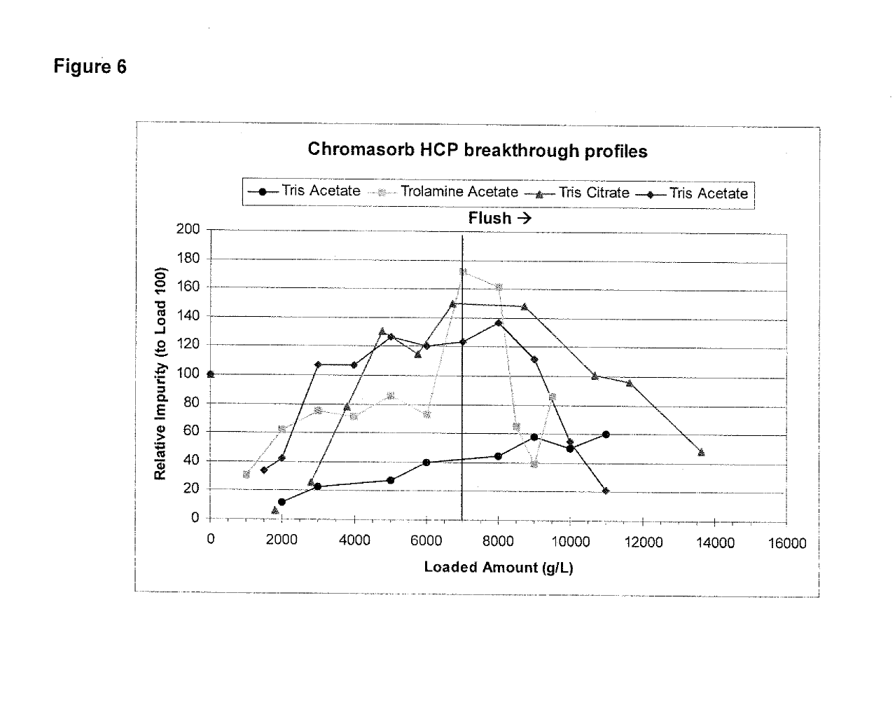

FIG. 7 depicts the impact of varying Tris concentration on aggregate reduction and recovery for selected cation exchange resins Nuvia S (panel A) and Capto S (panel B)) for antibody A. GigaCap S shows similar behavior to Capto S (data not shown). Elution recovery is shown with closed symbols and aggregate profile is shown with open symbols.

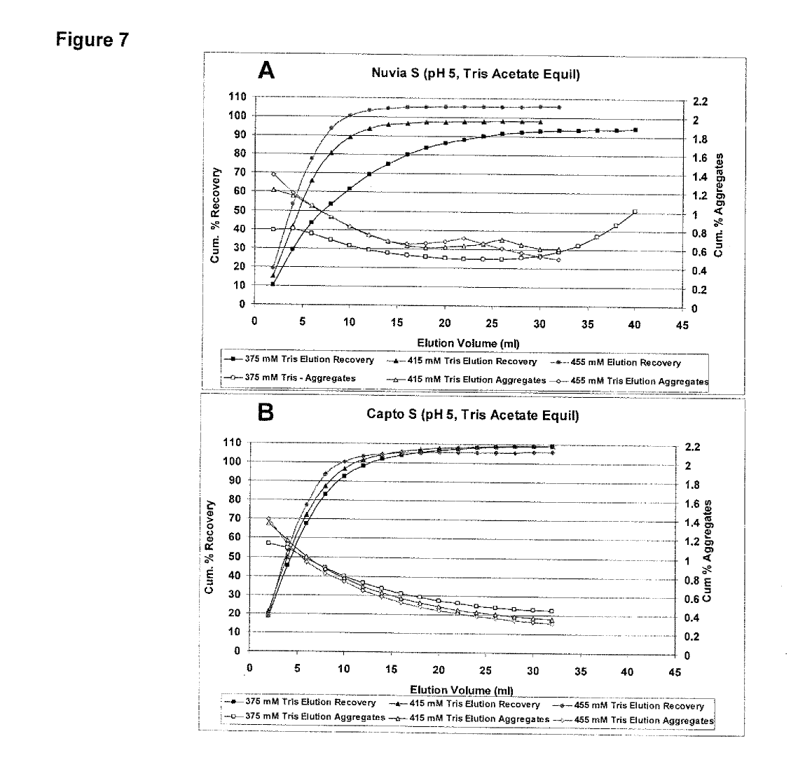

FIG. 8 depicts an example chromatogram overlay of blank runs showing pH and conductivity transition curves for Tris Acetate system for all three resins as compared to a no column run.

FIG. 9 depicts an example chromatogram overlay of blank runs (performed in the same manner as FIG. 11) showing a delayed pH and conductivity transition curves for the sodium phosphate buffer system.

FIG. 10 depicts an example chromatogram overlay of blank runs (performed in the same manner as FIG. 11) across different cations for the same anionic system illustrating that the transitions in pH and conductivity are independent of the cation.

FIG. 11 an example chromatogram overlay of blank runs (performed in the same manner as FIG. 11) across different anions for the same cationic system illustrating that the anion significantly affects the shape of the pH transition curve, especially during the elution step.

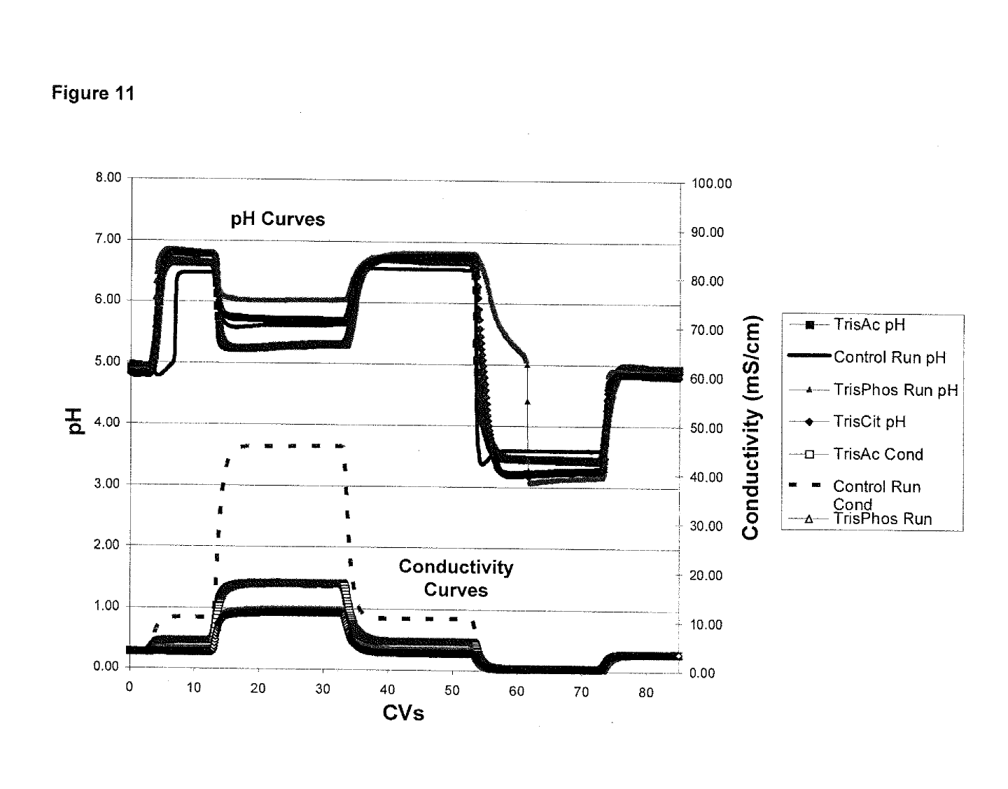

FIG. 12 depicts a contour plot of resultant HCP content after mAbSelect Sure capture for varying WashII cation concentrations and pH in selected two component buffer systems for antibody A.

FIG. 13 depicts a summary from a selected throughput screening study performed with pre-loaded PreDictor plates (GE Healthcare) to determine anion exchange resin performance for two molecules in Tris Acetate buffer. A range of pHs, conductivities and protein concentration were evaluated for recovery and impurity reduction from pH inactivated mAbSelect eluate.

FIG. 14 depicts the HCP reduction (expressed as reduction factor) of selected anion exchange resins and membranes for two molecules in Tris Acetate (pH 7.7, 2.5 mS/cm)

FIG. 15 depicts the impact of selected buffer systems on the HCP breakthrough profile from selected anion exchange membranes. Buffers used were two component buffer systems at pH 7.9. 4.5 mS/cm.

FIG. 16 depicts the impact of selected buffer systems on host cell protein (HCP) and aggregate breakthrough profiles for selected molecules using two components buffer systems at pH 7.9, 4.5 mS/cm.

### DETAILED DESCRIPTION OF THE INVENTION

The present invention is directed to the use of an integrated approach to purification process development. In certain embodiments, this approach concerns the development of processes comprising particular capture and fine purification steps. For example, in certain embodiments, the methods described herein can employ a single capture separation, such as a Protein A-based separation, followed by a single fine purification separation, such as a mixed mode separation. However, in certain embodiments, the fine purification step can include one, two, three or more individual separations, such as, but not limited to, a mixed mode separation (i.e., a separation that is based on more than one molecular or ionic interaction, such as by a separation based on a chromatographic media that is capable of ionic interactions, hydrogen bonding, and hydrophobic interactions) followed by a anion or cation exchange membrane-based separation.

Another aspect of the invention, the integrated approach to purification process development concerns the use of a minimum number of buffer systems during the course of the process. For example, in certain embodiments, a single buffer system is employed throughout the capture and fine purification steps. In particular embodiments the buffer system will consist essentially of water and two other ionic components, namely an anionic component and a cationic component, with the two ionic components being mixed in different combinations and concentrations to create buffers suitable for the needs of any particular purification process.

In a further aspect of the invention, the integrated approach to purification process development concerns the use of a minimally-corrosive, chloride free buffer system. Certain buffer systems employ chloride salts, which can have corrosive impact on commercial antibody production and purification equipment such as stainless steel. In certain embodiments, the present invention relates to buffer systems that employ minimally-corrosive buffer systems, such as, but not limited to, those that employ either Tris or Trolamine paired with either acetate (as acetic acid) or citrate (as citric acid). No counterion such as sodium are included.

In certain embodiments the present invention relates to purification processes where, one, two or all three aspects of the integrated approach to purification process development are employed. For example, in certain embodiments, a purification process of the present invention will comprise a single capture separation and single fine purification separation, where both separations employ the same buffer system. Further, in certain of such embodiments, that single buffer system will be a minimally-corrosive buffer system.

For clarity and not by way of limitation, this detailed description is divided into the following sub-portions:
1. Definitions;
2. Antibody Generation;
3. Antibody Expression;
4. Integrated Antibody Purification Steps;
5. Buffer Systems;
6. Minimally-Corrosive Buffer Systems; and
7. Exemplary Integrated Purification Strategies

### 1. DEFINITIONS

In order that the present invention may be more readily understood, certain terms are first defined.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or “antibody portion”) includes fragments of an antibody that retain the ability to specifically bind to an antigen (e.g., hIL-12, hTNFα, or hIL-18). It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “modifying”, as used herein, is intended to refer to changing one or more amino acids in the antibodies or antigen-binding portions thereof. The change can be produced by adding, substituting or deleting an amino acid at one or more positions. The change can be produced using known techniques, such as PCR mutagenesis.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The phrase “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”). Such decreases in the number and/or activity of viral particles can be on the order of about 1% to about 99.99999%, preferably of about 20% to about 99%, more preferably of about 30% to about 990, more preferably of about 40% to about 99%, even more preferably of about 50% to about 99%, even more preferably of about 60% to about 99%, yet more preferably of about 70% to about 99%, yet more preferably of about 80% to 99%, and yet more preferably of about 90% to about 99%. In certain non-limiting embodiments, the amount of virus, if any, in the purified antibody product is less than the ID50 (the amount of virus that will infect 50 percent of a target population) for that virus, preferably at least 10-fold less than the ID50 for that virus, more preferably at least 100-fold less than the ID50 for that virus, and still more preferably at least 1000-fold less than the ID50 for that virus. In certain embodiments, a method for quantifying is the log reduction in the infectivity or ability to replicate from about 0.5 log to 8 log reduction.

The phrase “contact position” includes an amino acid position in the CDR1, CDR2 or CDR3 of the heavy chain variable region or the light chain variable region of an antibody which is occupied by an amino acid that contacts antigen in one of the twenty-six known antibody-antigen structures. If a CDR amino acid in any of the twenty-six known solved structures of antibody-antigen complexes contacts the antigen, then that amino acid can be considered to occupy a contact position. Contact positions have a higher probability of being occupied by an amino acid which contact antigens than in a non-contact position. In one aspect, a contact position is a CDR position which contains an amino acid that contacts antigen in greater than 3 of the 26 structures (>1.5%). In another aspect, a contact position is a CDR position which contains an amino acid that contacts antigen in greater than 8 of the 25 structures (>32%).

### 2. ANTIBODY GENERATION

The antibodies of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One preferred animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody preferably can be a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one non-limiting embodiment, the antibodies of this disclosure are human monoclonal antibodies. Such human monoclonal antibodies can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen).

Moreover, alternative transchromosomic animal systems expressing human immunoglobulin genes are available in the art and can be used to raise antibodies of the disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise antibodies of this disclosure.

Recombinant human antibodies of the invention, or an antigen binding portion thereof, can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679: Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et at. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In yet another embodiment of the invention, the antibodies of the instant invention, or fragments thereof, can be altered wherein the constant region of the antibody is modified to reduce at least one constant region-mediated biological effector function relative to an unmodified antibody. To modify an antibody of the invention such that it exhibits reduced binding to the Fe receptor, the immunoglobulin constant region segment of the antibody can be mutated at particular regions necessary for Fe receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

### 3. ANTIBODY EXPRESSION

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. For example, one approach to converting the antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. No. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact antibodies, such as Fab fragments or scFv molecules. It is understood that variations on the above procedure are within the scope of the present invention. For example, in certain embodiments it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to the particular target antigen. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the initial target antigen by crosslinking an antibody of the invention to a second antibody by standard chemical crosslinking methods.

In a suitable system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. In one aspect, if the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### 4. INTEGRATED ANTIBODY PURIFICATION STEPS

### 4.1 Comparison of Integrated Approach and Traditional Antibody Purification

Traditional purification methods for producing a purified (Host Cell Protein—or “HCP-reduced”) antibody preparation from a mixture comprising an antibody and at least one HCP conventionally employ four steps: (1) harvest/clarification—separation of host cells from the fermentation culture; (2) capture—separation of antibody from the majority of components in the clarified harvest; (3) fine purification—separation of the antibody from residual HCP contaminants and aggregates; and (4) formulation—placing the antibody into an appropriate carrier for maximum stability and shelf life. Table 1 summarizes one embodiment of such a traditional a purification scheme. As outlined in that scheme, a variety of capture and fine purification separations are often employed in traditional purification processes in order produce an antibody composition substantially free of HCPs.

TABLE 1
Purification steps with their associated purpose

| Purification step | Purpose |
|---|---|
| Primary recovery | clarification of sample matrix |
| Cation exchange or | antibody capture, host cell protein and |
| Affinity chromatography | associated impurity reduction |
| ultrafiltration/diafiltration | concentration and buffer exchange |
| (if necessary) | |
| Viral Inactivation | Inactivate viruses by low pH treatment |
| Anion exchange or Mixed | reduction of host cell proteins and DNA |
| Mode chromatography | |
| Phenyl Sepharose ™ HP | reduction of antibody aggregates and host |
| chromatography or | cell proteins |
| Cation Exchange | |
| Chromatography | |
| Viral filtration | removal of viruses based on size, if present |
| Final | concentrate and formulate antibody |
| ultrafiltration/diafiltration | |

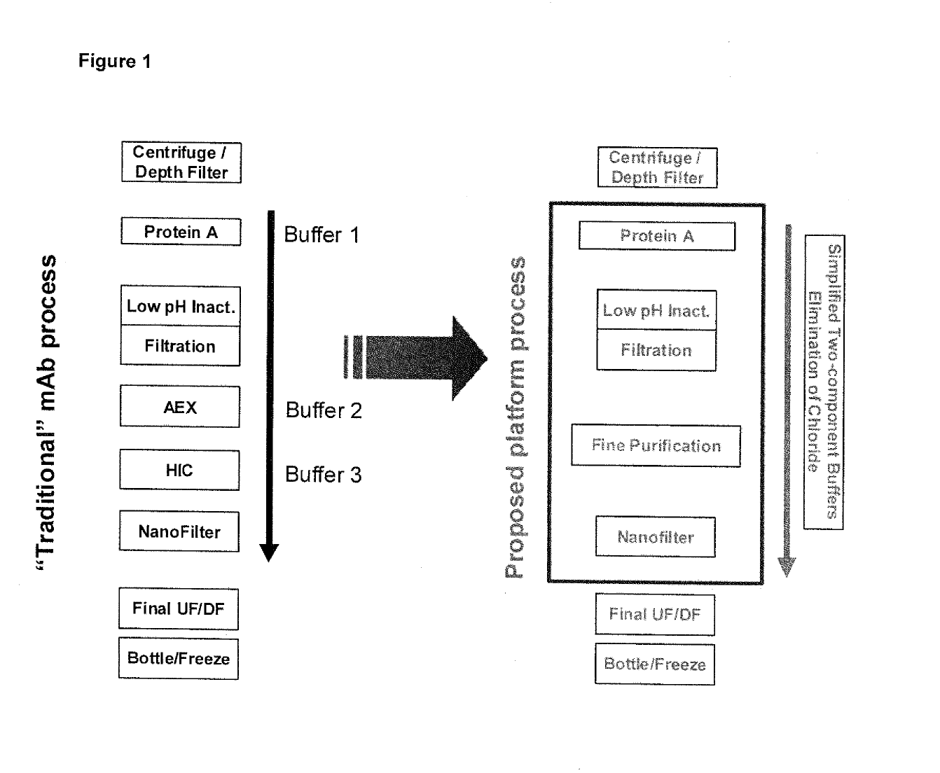

In contrast to traditional purification processes, which are conventionally designed from scratch for each individual antibody of interest, the present invention is directed, in certain embodiments, to the development of purification processes comprising steps that can be employed across a diverse genus of antibodies and yet still allow for effective HCP reduction. While the integrated approach tracks the traditional four steps of clarification, capture, fine purification, and formulation, the integrated approach allows for improvements in the resulting antibody's purity as well as in process design and run-time efficiencies by, in certain embodiments, minimizing the number of individual separations that occur at each step. A side-by-side comparison of a traditional purification process and an integrated process of the present invention is depicted in FIG. 1.

### 4.2 Clarification & Primary Recovery Step

The initial step of the purification methods of the present invention involve the clarification and primary recovery of antibody from a sample matrix. In addition, the primary recovery process can also be a point at which to reduce or inactivate viruses that can be present in the sample matrix. In the context of the instant integrated purification process, any one or more of a variety of methods of viral reduction/inactivation can be used during the primary recovery phase of purification including heat inactivation (pasteurization), pH inactivation, solvent/detergent treatment, UV and γ-ray irradiation and the addition of certain chemical inactivating agents such as β-propiolactone or e.g., copper phenanthroline as in U.S. Pat. No. 4,534,972, the entire teaching of which is incorporated herein by reference. In certain embodiments of the present invention, the sample matrix is exposed to pH viral reduction/inactivation during the primary recovery phase.

Methods of pH viral reduction/inactivation include, but are not limited to, incubating the mixture for a period of time at low pH, and subsequently neutralizing the pH and removing particulates by filtration. In certain embodiments the mixture will be incubated at a pH of between about 2 and 5, preferably at a pH of between about 3 and 4, and more preferably at a pH of about 3.5. The pH of the sample mixture may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus reduction/inactivation is affected by pH and the duration of the low pH incubation. In certain embodiments the duration of the low pH incubation will be from 0.5 hr to two 2 hr, preferably 0.5 hr to 1.5 hr, and more preferably the duration will be 1 hr. Virus reduction/inactivation is dependent on these same parameters in addition to protein concentration, which may limit reduction/inactivation at high concentrations. Thus, the proper parameters of protein concentration, pH, and duration of reduction/inactivation can be selected to achieve the desired level of viral reduction/inactivation.

In certain embodiments viral reduction/inactivation can be achieved via the use of suitable filters. A non-limiting example of a suitable filter is the Ultipor DV50™ filter from Pall Corporation. Although certain embodiments of the present invention employ such filtration during the capture phase, in other embodiments it is employed at other phases of the purification process, including as either the penultimate or final step of purification. In certain embodiments, alternative filters are employed for viral reduction/inactivation, such as, but not limited to, Ultipor DV20™ filter from Pall Corporation; ViroSart CPV from Sartorius; Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, Ill.).

In those embodiments where viral reduction/inactivation is employed, the sample mixture can be adjusted, as needed, for further purification steps. For example, following low pH viral reduction/inactivation the pH of the sample mixture is typically adjusted to a more neutral pH, e.g., from about 4.5 to about 8.5, and preferably about 4.9, prior to continuing the purification process. Additionally, the mixture may be diluted with water for injection (WFI) to obtain a desired conductivity.

In certain embodiments, the primary recovery will include one or more centrifugation steps to further clarify the sample matrix and thereby aid in purifying the antibodies of interest. Centrifugation of the sample can be run at, for example, but not by way of limitation, 7,000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification.

In certain embodiments, the primary recovery will include the use of one or more depth filtration steps to further clarify the sample matrix and thereby aid in purifying the antibodies of the present invention. Depth filters contain filtration media having a graded density. Such graded density allows larger particles to be trapped near the surface of the filter while smaller particles penetrate the larger open areas at the surface of the filter, only to be trapped in the smaller openings nearer to the center of the filter. In certain embodiments the depth filtration step can be a delipid depth filtration step. Although certain embodiments employ depth filtration steps only during the primary recovery phase, other embodiments employ depth filters, including delipid depth filters, during one or more additional phases of purification. Non-limiting examples of depth filters that can be used in the context of the instant invention include the Cuno™ model 30/60ZA depth filters (3M Corp.), and 0.45/0.2 μm Sartopore™ bi-layer filter cartridges.

### 4.3 Capture Step

In certain embodiments of the present invention, the primary recovery sample is subjected to a capture step to further purify the antibody of interest away from the fermentation media containing HCPs. In certain embodiments the capture step employs chromatographic material that is capable of selectively or specifically binding to the antibody of interest. Non-limiting examples of such chromatographic material include: Protein A, Protein G, chromatographic material comprising the antigen bound by the antibody of interest, and chromatographic material comprising an Fc binding protein.

In specific embodiments of the present invention, the capture step involves subjecting the primary recovery sample to a column comprising a suitable Protein A resin. Protein A resin is useful for affinity purification and isolation of a variety antibody isotypes, particularly IgG₁, IgG₂, and IgG₄. Protein A is a bacterial cell wall protein that binds to mammalian IgGs primarily through their Fe regions. In its native state, Protein A has five IgG binding domains as well as other domains of unknown function.

There are several commercial sources for Protein A resin. One suitable resin is MabSelect™ from GE Healthcare. Other suitable Protein A resins include, but are not limited to: mAbSelect SurRe™ from GE Healthcare and ProSep Ultra Plus™ from Millipore. A non-limiting example of a suitable column packed with MabSelect™ is an about 1.0 cm diameter×about 21.6 cm long column (˜17 mL bed volume). This size column can be used for small scale purifications and can be compared with other columns used for scale ups. For example, a 20 cm×21 cm column whose bed volume is about 6.6 L can be used for larger purifications. Regardless of the column, the column can be packed using a suitable resin such as MabSelect™.

As discussed in detail in Sections 5 and 6, below, in certain embodiments, the present invention relates to purification process that use of a minimum number of buffer systems during the course of the capture step as well as buffers that comprise minimally-corrosive components. For example, in certain embodiments, a single buffer system is employed throughout the capture step. In particular embodiments the buffer system will consist of only water and two other ionic components, namely an anionic component and a cationic component, with the two ionic components being mixed in different combinations and concentrations to create buffers suitable for the needs of any particular purification process. In certain embodiments, the buffer system employed in the context of the capture step employs a Tris or a Trolamine component. In certain embodiments, the buffer system employed in the context of the capture step employs an acetate or citrate component. In particular embodiments, the buffer system employed in the context of the capture step is a Tris-acetate buffer, a Tris-citrate buffer, a Trolamine-acetate buffer, or a Trolamine-citrate buffer.

### 4.4 Fine Purification Step

In addition to the capture step, the integrated purification process of the present invention comprise, in certain embodiments, a fine purification step. In certain embodiments, the fine purification step comprises a single separation, such as, but not limited to an ion exchange-based separation, a hydrophobic interaction-based separation, or a mixed mode-based separation. In certain embodiments, the fine purification step comprises two, three, four, or more individual separations. For example, but not by way of limitation, the fine purification step can comprise a mixed mode-based chromatographic separation followed by an ion exchange separation. In particular non-limiting embodiments, the fine purification step comprises a mixed mode-based separation using CaptoAdhere™ resin followed by either an anion exchange chromatographic separation or an anion exchange membrane separation, such as a ChromaSorb™ Q membrane. In particular non-limiting embodiments, the fine purification step comprises a mixed mode-based separation using CaptoAdhere™ resin or anion exchange separation followed by either a cation exchange separation or a hydrophobic interaction separation.

As discussed in detail in Sections 5 and 6, below, in certain embodiments, the present invention relates to purification process that use of a minimum number of buffer systems during the course of the fine purification step as well as buffers that comprise minimally-corrosive components. For example, in certain embodiments, a single buffer system is employed throughout the fine purification step. In particular embodiments the buffer system will consist of only water and two other ionic components, namely an anionic component and a cationic component, with the two ionic components being mixed in different combinations and concentrations to create buffers suitable for the needs of any particular purification process. In certain embodiments, the buffer system employed in the context of the fine purification step employs a Tris or a Trolamine component. In certain embodiments, the buffer system employed in the context of the fine purification step employs an acetate or citrate component. In particular embodiments, the buffer system employed in the context of the fine purification step is a Tris-acetate buffer, a Tris-citrate buffer, a Trolamine-acetate buffer, or a Trolamine-citrate buffer.

### 4.4.1 Ion Exchange Separations

In certain embodiments, the instant invention provides methods for producing a HCP-reduced antibody preparation from a mixture comprising an antibody and at least one HCP by subjecting the mixture to at least one ion exchange separation such that an eluate comprising the antibody is obtained. Ion exchange separation includes any method by which two substances are separated based on the difference in their respective ionic charges, and can employ either cationic exchange material or anionic exchange material.

In certain embodiments, the sample from a first fine purification ion exchange separation is subjected to a second ion exchange separation. Preferably this second ion exchange separation will involve separation based on the opposite charge of the first ion exchange separation. For example, if an anion exchange step is employed first, the second ion exchange chromatographic step can be a cation exchange step. Conversely, if the first ion exchange separation was a cation exchange separation, that step would be followed by an anion exchange separation. The use of a cationic exchange material versus an anionic exchange material is based on the overall charge of the protein and whether the intention is to perform the separation by retaining the antibody of interest on the column and allow the HCPs to flow through or, conversely, to retain the HCPs on the column and allow the antibody of interest to flow through.

In performing the separation, the initial antibody mixture can be contacted with the ion exchange material by using any of a variety of techniques, e.g., using a batch purification technique or a chromatographic technique. For example, in the context of batch purification, ion exchange material is prepared in, or equilibrated to, the desired starting buffer. Upon preparation, or equilibration, a slurry of the ion exchange material is obtained. The antibody solution is contacted with the slurry to adsorb the antibody to be separated to the ion exchange material. The solution comprising the HCP(s) that do not bind to the ion exchange material is separated from the slurry, e.g., by allowing the slurry to settle and removing the supernatant. The slurry can be subjected to one or more washes. If desired, the slurry can be contacted with a solution of higher conductivity to desorb HCPs that have bound to the ion exchange material. In order to elute bound polypeptides, the salt concentration of the buffer can be increased.

Ion exchange chromatography can also be used as an ion exchange separation technique. Ion exchange chromatography separates molecules based on differences between the overall charge of the molecules. For the purification of an antibody, the antibody must have a charge opposite to that of the functional group attached to the ion exchange material, e.g., resin, in order to bind. For example, antibodies, which generally have an overall positive charge in the buffer pH below its pI, will bind well to cation exchange material, which contain negatively charged functional groups.

In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAE), quaternary aminoethyl(QAE) and quaternary amine(Q) groups, and which are used in commercially available products such as, but not limited to Capto-Q™ (GE Healthcare), Toyopearl QAE55™ (Toso Haas Co.), Poros 50HQ™ and Poros 50PI™ (Applied Biosystems). Cationic substitutents include carboxymethyl (CM), sulfoethyl(SE), sulfopropyl(SP), phosphate(P) and sulfonate(S) and which are used in commercially available produces such as, but not limited to Capto-S™ (GE Healthcare), Gigacap-S™ (Toso Haas Co.), and Nuvia-S™ (BioRad). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from Pharmacia AB. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa.

In certain embodiments, a mixture comprising an antibody of interest and impurities, e.g., HCP(s), is loaded onto an ion exchange column, such as a cation exchange column. For example, but not by way of limitation, the mixture can be loaded at a load of about 80 g protein/L resin depending upon the column used. An example of a suitable cation exchange column is a 80 cm diameter×23 cm long column whose bed volume is about 116 L. The mixture loaded onto this cation column can subsequently washed with wash buffer (equilibration buffer). The antibody is then eluted from the column, and a first eluate is obtained.

### 4.4.2 Hydrophobic Interaction Separations

The present invention also features methods for producing a HCP-reduced antibody preparation from a mixture comprising an antibody and at least one HCP comprising a hydrophobic interaction separation. For example, in certain embodiments, a first eluate obtained from a capture step can be subjected to a hydrophobic interaction material such that a second eluate having a reduced level of HCP is obtained. In alternative embodiments, a HIC separation is employed as a second, third, or subsequent separation in the context of the fine purification step. Hydrophobic interaction chromatography steps, such as those disclosed herein, are generally performed to remove protein aggregates, such as antibody aggregates, and process-related impurities.

In performing a HIC separation, the sample mixture is contacted with the HIC material, e.g., using a batch purification technique or using a column. Prior to HIC separation it may be desirable to remove any chaotropic agents or very hydrophobic substances, e.g., by passing the mixture through a pre-column.

For example, in the context of batch purification, HIC material is prepared in or equilibrated to the desired equilibration buffer. A slurry of the HIC material is obtained. The antibody solution is contacted with the slurry to adsorb the antibody to be separated to the HIC material. The solution comprising the HCPs that do not bind to the HIC material is separated from the slurry, e.g., by allowing the slurry to settle and removing the supernatant. The slurry can be subjected to one or more washes. If desired, the slurry can be contacted with a solution of lower conductivity to desorb antibodies that have bound to the HIC material. In order to elute bound antibodies, the salt concentration can be decreased.

Whereas ion exchange chromatography relies on the charges of the antibodies to isolate them, hydrophobic interaction chromatography (HIC) depends on the hydrophobic properties of the antibodies. Hydrophobic groups on the antibody interact with hydrophobic groups on the column. The more hydrophobic a protein is, the stronger it will interact with the column. Thus the HIC separation is capable of removing host cell-derived impurities (e.g., DNA and other high and low molecular weight product-related species).

Hydrophobic interactions are strongest at high ionic strength, therefore, this form of separation is conveniently, though not exclusively, performed following salt precipitations or ion exchange procedures. Adsorption of the antibody to a HIC column is favored by high salt concentrations, but the actual concentrations can vary over a wide range depending on the nature of the antibody and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba⁺⁺; Ca⁺⁺; Mg⁺⁺; Li⁺; Cs⁺; Na⁺; K⁺; Rb⁺; NH₄⁺, while anions may be ranked in terms of increasing chaotropic effect as P0⁻⁻; S0₄⁻⁻; CH₃CO₃⁻; Cl⁻; Br⁻; NO₃⁻; ClO₄⁻; I⁻; SCN⁻. In certain embodiments the anion is C₃H₅O(COO)3³⁻.

In general, Na, K or NH₄ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful. It is sufficient that one component of the “solution”, anion or cation, is hydrophobic interaction promoting. One non-limiting example is Tris-Citrate where citrate is effective in promoting hydrophobic interactions.

HIC columns normally comprise a base matrix (e.g., cross-linked agarose or synthetic copolymer material) to which hydrobobic ligands (e.g., alkyl or aryl groups) are coupled. A suitable HIC column comprises an agarose resin substituted with phenyl groups (e.g., a Phenyl Sepharose™ column). Many HIC columns are available commercially. Examples include, but are not limited to, Phenyl Sepharose™ 6 Fast Flow column with low or high substitution (Pharmacia LKB Biotechnology, AB, Sweden); Phenyl Sepharose™ High Performance column (Pharmacia LKB Biotechnology, AB, Sweden); Octyl Sepharose™ High Performance column (Pharmacia LKB Biotechnology, AB, Sweden); Fractogel™ EMD Propyl or Fractogel™ EMD Phenyl columns (E. Merck, Germany); Macro-Prep™ Mehyl or Macro-Prep™ t-Butyl Supports (Bio-Rad, California); WP HI-Propyl (C3)™ column (J. T. Baker, New Jersey); and Toyopearl™ ether, phenyl or butyl columns (Tosollaas, PA).

### 4.4.3 Mixed Mode Separations

The present invention also features methods for producing a HCP-reduced antibody preparation from a mixture comprising an antibody and at least one HCP comprising a mixed mode separation. For example, in certain embodiments, a first eluate obtained from a capture step can be subjected to a mixed mode separation material such that a second eluate having a reduced level of HCP is obtained. In alternative embodiments, a mixed mode separation is employed as a second, third, or subsequent separation in the context of the fine purification step.

Non-limiting examples of commercially available mixed-mode resins include: MEP-Hypercel™ (Pall Corp.); Capto-MMC™ (GE Healthcare); and Capto-Adhere™ (GE Healthcare). In particular, non-limiting, embodiments the process of the present invention includes a fine purification step employing a Capto-Adhere™ (GE Healthcare)-based separation. Capto-Adhere™ (GE Healthcare) is a highly cross-linked agarose with a ligand (N-Benzyl-N-methyl ethanol amine) that exhibits multiple functionalities for interaction, such as ionic interaction, hydrogen bonding, and hydrophobic interaction.

In certain embodiments of the present invention a fine purification step employing a Capto-Adhere™ (GE Healthcare)-based separation is performed according to the following conditions: resin—4.7 mL HiScreen Capto-Adhere™ (GE Healthcare); Tris-acetate buffer; pH 7.0-8.2; conductivity 4-12 mS/cm; antibody load up to 300 g/L resin; pH adjusted using 3M Tris or 3M acetic acid; conductivity adjusted with the concentration of Tris-acetate. In particular non-limiting examples, the conductivity is maintained at 4.0-5.0 mS/cm, the pH is maintained at 7.8-8.0, and antibody load is limited to 150-200 g/L resin.

### 4.5 Ultrafiltration/Diafiltration & Viral Inactivation

Certain embodiments of the present invention employ ultrafiltration and/or diafiltration steps to further purify and concentrate the antibody sample. As outlined in FIG. 1, such UF/DF and viral inactivation steps can occur multiple times and at various times during the course of a purification process. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 μm. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultratfiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody preparations.

### 5. BUFFER SYSTEMS

In another aspect of the invention, the integrated approach to purification process development concerns the use of a minimal number of buffer systems. In certain embodiments, this minimal number of buffer systems occurs in the context of the entire purification process. In certain embodiments, it occurs in the context of the capture and fine purification steps. For example, in certain embodiments, a single buffer system is employed throughout the capture and fine purification steps. In particular embodiments, the buffer system will consist of only water and two other ionic components, such as an anionic component and a cationic component. In certain embodiments the two ionic components are mixed in different combinations and concentrations to create buffers suitable for the needs of any particular purification process, typically from pH ˜3 to pH ˜8. In certain embodiments, additional components can be incorporated into the buffer system, such as metal chelators and/or protease inhibitors.

In certain embodiments of the present invention the use of particular buffer components, for example in a single buffer purification scheme, allows for control over pH and conductivity. pH control is achieved by titrating either the anion (lower pH) or cation (higher pH) with the corresponding ionic component. Conductivity is controlled by the concentration of components, eliminating addition of other ionic components, typically sodium chloride. As outlined in the Examples, below, the adjustment of such components and resulting control of pH and conductivity can impact impurity, HCP, and/or aggregate removal, as well as improving product recovery.

In certain embodiments, the buffer system will incorporate an equilibration buffer comprising, for example, 25 mM Tris-Acetate pH 7.2; 25 mM Trolamine-Acetate pH 7.2; 25 mM Tris-Citrate pH 7.2; 25 mM Trolamine-Citrate pH 7.2; 25 mM Tris-Phosphate pH 7.2; 25 mM Trolamine-Phosphate pH 7.2; 25 mM sodium-phosphate, pH 7.2; 25 mM sodium-Citrate pH 7.2; 140 mM Tris-123 mM Acetate pH 7.2; 150 mM Trolamine-120 mM Acetate pH 7.2; 70 mM Tris-21 mM Citrate pH 7.2; 80 mM Trolamine-22 mM Citrate pH 7.2; 105 mM Tris-62 mM Phosphate pH 7.2; 115 mM Trolamine-62 mM Phosphate pH 7.2; or 90 mM Na-60 mM phosphate, pH 7.2. In certain embodiments, the buffer system will incorporate a wash buffer comprising, for example, 25 mM Tris-Acetate pH 7.2; 25 mM Trolamine-Acetate pH 7.2; 25 mM Tris-Citrate pH 7.2; 25 mM Trolamine-Citrate pH 7.2; 25 mM Tris-Phosphate pH 7.2; 25 mM Trolamine-Phosphate pH 7.2; 25 mM sodium-phosphate, pH 7.2; 25 mM sodium-Citrate pH 7.2; 590 mM Tris-655 mM Acetate pH 5.7; 595 mM Trolamine-658 mM Acetate pH 5.7; 355 mM Tris-158 mM Citrate pH 6.0; 360 mM Trolamine-159 mM Citrate pH 6.0; 545 mM Tris-483 mM Phosphate pH 6.3; 555 mM Trolamine-485 mM Phosphate pH 6.3; 535 mM Na-482 mM Phosphate pH 6.3; 300 mM Tris-Acetate pH 7.2; 300 mM Trolamine-Acetate pH 7.2; or 300 mM Trolamine-Citrate pH 7.2. In certain embodiments, the buffer system will incorporate an elution buffer comprising, for example, 100 mM sodium Acetate pH 3.5; 100 mM Acetate (5 mM Tris) pH 3.5; 100 mM Acetate (5 mM Trolamine) pH 3.5; 25 mM sodium acetate pH 3.5, 25 mM Acetate (1 mM Tris) pH 3.5, 25 mM Acetate (1 mM Trolamine) pH 3.5, 25 mM Citrate (5 mM Tris) pH 3.5; 25 mM Citrate (5 mM Trolamine) pH 3.5; 25 mM Phosphate (Tris) pH 3.2; 25 mM Phosphate (Trolamine) pH 3.2; 25 mM Phosphate (sodium) pH 3.2; 25 mM Citric Acid (sodium citrate) pH 3.5; 100 mM Acetate (4.89 mM Tris) pH 3.5; 100 mM Acetate (4.89 mM Trolamine) pH 3.5; 25 mM Citric (Tris) Acid pH 3.5; 25 mM Citric (Trolamine) Acid pH 3.5; 25 mM Acetate (0.89 mM Tris) pH 3.5; 25 mM Acetate (0.89 mM Trolamine) pH 3.5; 7.5 mM Citric (5.3 mM Tris) Acid pH 3.5; 7.5 mM Citric (5.3 mM Trolamine) Acid pH 3.5; 5 mM Phosphate (4.0 mM Tris) pH 3.2; 5 mM Phosphate (4.0 mM Trolamine) pH 3.2; or 5 mM Phosphate (4.0 mM Sodium) pH 3.2.

In certain embodiments, the buffer system will comprise water and Tris-Citrate, Trolamine-Citrate, Tris-Acetate, or Trolamine-Acetate at a pH of 7.0-8.2; and a conductivity: 2-12 mS/cm. In certain embodiments, the buffer system will comprise water and Tris-Citrate, Trolamine-Citrate, Tris-Acetate, or Trolamine-Acetate at a pH of 7.9±0.1 and a conductivity of 4.5±0.5 mS/cm. In alternative embodiments, the buffer system will comprise water and Tris-Citrate, Trolamine-Citrate, Tris-Acetate, or Trolamine-Acetate at a pH of 7.7±0.1, and conductivity of 2.5±0.5 mS/cm.

### 6. MINIMALLY-CORROSIVE BUFFER SYSTEMS

In a further aspect of the invention, the integrated approach to purification process development concerns the use of a minimally-corrosive, chloride free buffer system. Certain buffer systems employ chloride salts that can have corrosive impact on commercial antibody production and purification equipment, such as stainless steel. Accordingly, buffers comprising chloride salts or similarly corrosive alternatives are generally, although not uniformly, excluded from the scope of the instant invention.

In certain embodiments, the present invention relates to buffer systems that employ minimally-corrosive buffer systems, such as, but not limited to, those that employ either Tris or Trolamine paired with either acetate or citrate. For example, certain non-limiting embodiments the buffer system will comprise Tris-acetate, Tris-citrate, Trolamine-acetate, or Trolamine citate.

### 7. EXEMPLARY INTEGRATED PURIFICATION STRATEGIES

In certain embodiments of the present invention, the integrated purification strategy comprises the following four steps: (1) harvest/clarification—separation of host cells from the fermentation culture; (2) capture—separation of antibody from the majority of components in the clarified harvest; (3) fine purification—separation of the antibody from residual host cell contaminants and aggregates; and (4) formulation.

In certain embodiments the harvest/clarification step is accomplished by centrifugation and/or depth filtration. Non-limiting examples of depth filters useful in the context of such harvest/clarification steps include: Cuno™ model 30/60ZA depth filters (3M Corp.), and 0.45/0.2 μm Sartopore™ bi-layer filter cartridges.

In certain embodiments, the capture and fine purification steps is accomplished by a two-column procedure. For example, but not by way of limitation, a Protein A-based capture step can be combined with a mixed mode-based fine purification step. In particular non-limiting examples, the Protein A-based step employs MabSelect™ from GE Healthcare, mAbSelect SuRe™ from GE Healthcare, or ProSep Ultra Plus™ from Millipore. In certain embodiments the mixed mode-based step employs Capto-Adhere™ (GE Healthcare).

In certain embodiments, a two column strategy can be supplemented by the presence of one or more filtration separations and or one or more viral inactivation steps. In certain embodiments the capture step is followed by a viral inactivation step, such as, but not limited to, a low pH viral inactivation step, and a filtration step, such as, but not limited to, an F0HC (Millipore) filtration step.

In certain embodiments the fine purification step can be supplemented by the presence of one or more additional separations, such as, but not limited to an ion exchange separation or a hydrophobic interaction separation. In certain embodiments, such supplemental separations are selected from the group consisting of Chromosorb Q-based separations and Phenyl HP-based separations.

Particular examples of integrated purification processes include, but are not limited to, those that incorporate following:
MabSelect SuRe—F0HC—CaptoAdhere—ChromaSorb Q—ViroSart;
Prosep Ultra Plus—F0HC—CaptoAdhere—ChromaSorb Q—ViroSart;
MabSelect SuRe—F0HC—Nuvia S—ChromaSorb Q—ViroSart;
Prosep Ultra Plus—F0HC—Nuvia S—ChromaSorb Q—ViroSart; and
MabSelect SuRe—F0HC—Q Sepharose—Phenyl HP Sepharose.

In certain embodiment, such purification processes can also comprise a nanofiltration step, an ultrafiltration/diafiltration step, and a formulation (bottling/freezing) step.

### EXAMPLES

### 1. Capture Step

Prior to using actual clarified harvest material in the context of a capture step purification process, blank runs tracing pH and conductivity transitions across three Protein A resins evaluated were performed for the buffer systems being evaluated for effectiveness. The three resins evaluated were: Prosep Ultra Plus; Mabselect; and Mabselect Sure. Three anionic components were evaluated: Acetate; Citrate; and Phosphate. The three cationic components were evaluated: Sodium; Tris; and Trolamine. As discussed in detail below, the following resultant buffer systems were evaluated for the blank runs: conventional Protein A buffer system comprised of multiple components (Control); Tris Acetate; Tris Citrate; Tris Phosphate; Trolamine Acetate; Trolamine Citrate; Trolamine Phosphate; and Sodium Phosphate.

For the first set of blank runs, on the basis of comparability, buffers of similar ionic strength for each buffer system were used for the equilibration, wash and elution steps in the Protein A capture process, resulting in the following buffers shown in Table 2 shows the buffers that were used for each of the buffer systems evaluated. The concentrations used were determined using an ionic strength calculator which uses the Davis Equation as a basis for determining ionic concentrations.

TABLE 2
Buffers Used for Preliminary Blank Runs

| System | Equilibration | Wash | Elution |
|---|---|---|---|
| Control | 25 mM Tris + | 20 mM NaCitrate, | 100 mM |
| (conven- | 100 mM NaCl | 500 mM NaCl | NaAcetate |
| tional | pH 7.2* | pH 6.0 | pH 3.5* |
| buffer) | | | |
| Tris | 140 mM Tris- | 590 mM Tris | 100 mM Acetate |
| Acetate | 123 mM Acetate | 655 mM Acetate | (4.89 mM Tris) |
| | pH 7.2 | pH 5.7 | pH 3.5 |
| Trolamine | 150 mM Trol- | 595 mM Trol- | 100 mM Acetate |
| Acetate | 120 mM Acetate | 658 mM Acetate | (4.89 mM Trol) |
| | pH 7.2 | pH 5.7 | pH 3.5 |
| Tris | 70 mM Tris- | 355 mM Tris | 7.5 mM Citric |
| Citrate | 21 mM Citrate | 158 mM Citrate | (5.3 mM Tris) |
| | pH 7.2 | pH 6.0 | Acid pH 3.5 |
| Trolamine | 80 mM Trol- | 360 mMTrol | 7.5 mM Citric |
| Citrate | 22 mM Citrate | 159 mM Citrate | (5.3 mM Trol) |
| | pH 7.2 | pH 6.0 | Acid pH 3.5 |
| Tris | 105 mM Tris- | 545 mM Tris | 5 mM Phosphate |
| Phosphate | 62 mM Phosphate | 483 mM Phosphate | (4.0 mM Tris) |
| | pH 7.2 | pH 6.3 | pH 3.2 |
| Trolamine | 11.5 mM Trol- | 555 mM Trol | 5 mM Phosphate |
| Phosphate | 62 mM Phosphate | 485 mM Phosphate | (4.0 mM Trol) |
| | pH 7.2 | pH 6.3 | pH 3.2 |
| Sodium | 90 mM Na— | 535 mM Na | 5 mM Phosphate |
| Phosphate | 60 mM phosphate, | 482 mM Phosphate | (4.0 mM Na) |
| | pH 7.2 | pH 6.3 | pH 3.2 |

To evaluate the pH and conductivity transitions across the Protein A resins for each buffer system, the blank runs were structured similarly to the conventional Protein A capture process, wherein equilibration buffer is first run through each column (initially in storage buffer) until the pH and conductivity readings reach equilibrium (10 column volumes (CVs)) to simulate the equilibration, loading and Wash I steps. This is then followed by 20 CVs of wash buffer simulating the Wash II step. 20 CVs of equilibration buffer simulates the Wash III step, followed by 20 CVs of elution buffer. The columns are then put back into storage using 15 CVs of storage buffer (50 mM sodium acetate, 2% benzyl alcohol, pH 5.0). All process steps were run at 2 minute residence times. Similar runs were also performed where no columns were used as a negative control for column effect evaluation. The resultant chromatograms were then overlaid to evaluate the differences between the columns and the buffer systems.

For the second set of blank runs, the effect of post-storage regeneration on the volume of equilibration buffer needed to equilibrate the column before loading, and the effect of changing elution buffer concentrations on pH and conductivity transitions were evaluated. 0.2M acetic acid is generally used for regeneration of the Mabselect resins, while 0.15M phosphoric acid is used for the same purpose on PUP. For these set of runs, both regeneration buffers were evaluated on all 3 resins, and only the Tris containing buffer systems were evaluated. The following elution anionic concentrations, shown in Table 3, were used for the second set of blank runs. In this set of experiments, the columns were transferred from storage into 10 CVs of post-storage regeneration buffer followed by 10 CVs of water rinse to simulate the post-storage regeneration process, then proceed to 20 CVs each of equilibration and elution, and finally 15 CVs of storage buffer.

TABLE 3
Elution Anionic Concentrations for 2^{nd} Set of Blank Runs

| | Preliminary | Run 1 | Run 2 |
|---|---|---|---|
| | Anion Conc | Anion Conc | Anion Conc |
| System | (mM) | (mM) | (mM) |
| Control | 100 | 100 | 100 |
| Tris | 100 | 100 | 50 |
| Acetate | | | |
| Tris | 7.5 | 15 | 30 |
| Citrate | | | |
| Tris | 5 | 25 | 50 |
| Phosphate | | | |

After evaluating the effects of the buffers used on the resins, the next step who to evaluate the effects off the buffers on Protein A capture performance when clarified harvest material is loaded onto the resins. A 9th buffer system, the sodium citrate system, was evaluated in this set of runs. For these runs, all process steps were performed at 3 minute residence times. There were no Wash II steps used in these runs, meaning that only 10 CVs of equilibration buffer was used as the wash phase, and 0.15M phosphoric acid was used as the regeneration buffer for all 3 resins. Loading for all 3 resins was fixed at 30 g Antibody/L resin. Other than these conditions, the Mabselect and Mabselect Sure runs were performed similarly to conventional processes, except that the equilibration and elution buffers were changed whenever a different buffer system was run. For the Prosep Ultra Plus resin, the runs were performed similarly to the Mabselect runs, except that the equilibration and wash steps were performed at 1.6 minute residence times, and the Clean II and Sanitization steps were removed as well. The equilibration and elution buffers used for each of the buffer systems are shown in Table 4.

TABLE 4
Equilibration and Elution Buffers Used for Baseline Runs

| System | Equilibration | Wash | Elution |
|---|---|---|---|
| Control | 25 mM Tris + | 20 mM NaCitrate, | 100 mM |
| (Conven- | 100 mM NaCl | 500 mM NaCl | NaAcetate |
| tional | pH 7.2* | pH 6.0 | pH 3.5* |
| Buffer) | | | |
| Tris | 25 mM Tris- | 25 mM Tris- | 100 mM Acetate |
| Acetate | Acetate | Acetate | (4.89 mM Tris) |
| | pH 7.2 | pH 7.2 | pH 3.5 |
| Trolamine | 25 mM Trol- | 25 mM Trol- | 100 mM Acetate |
| Acetate | Acetate | Acetate | (4.89 mM Trol) |
| | pH 7.2 | pH 7.2 | pH 3.5 |
| Tris | 25 mM Tris- | 25 mM Tris- | 25 mM Citric |
| Citrate | Citrate | Citrate | (Tris) Acid |
| | pH 7.2 | pH 7.2 | pH 3.5 |
| Trolamine | 25 mM Trol- | 25 mM Trol- | 25 mM Citric |
| Citrate | Citrate | Citrate | (Trol) Acid |
| | pH 7.2 | pH 7.2 | pH 3.5 |
| Tris | 25 mM Tris- | 25 mM Tris- | 25 mM Phosphate |
| Phosphate | Phosphate | Phosphate | (Tris) pH 3.2 |
| | pH 7.2 | pH 7.2 | |
| Trolamine | 25 mM Trol- | 25 mM Trol- | 25 mM Phosphate |
| Phosphate | Phosphate | Phosphate | (Trol) pH 3.2 |
| | pH 7.2 | pH 7.2 | |
| Sodium | 25 mM Na- | 25 mM Na- | 25 mM Phosphate |
| Phosphate | phosphate, | phosphate, | (Na) pH 3.2 |
| | pH 7.2 | pH 7.2 | |
| Sodium | 25 mM Na- | 25 mM Na- | 25 mM Citric |
| Citrate | Citrate | Citrate | (Na) Acid |
| | pH 7.2 | pH 7.2 | pH 3.5 |

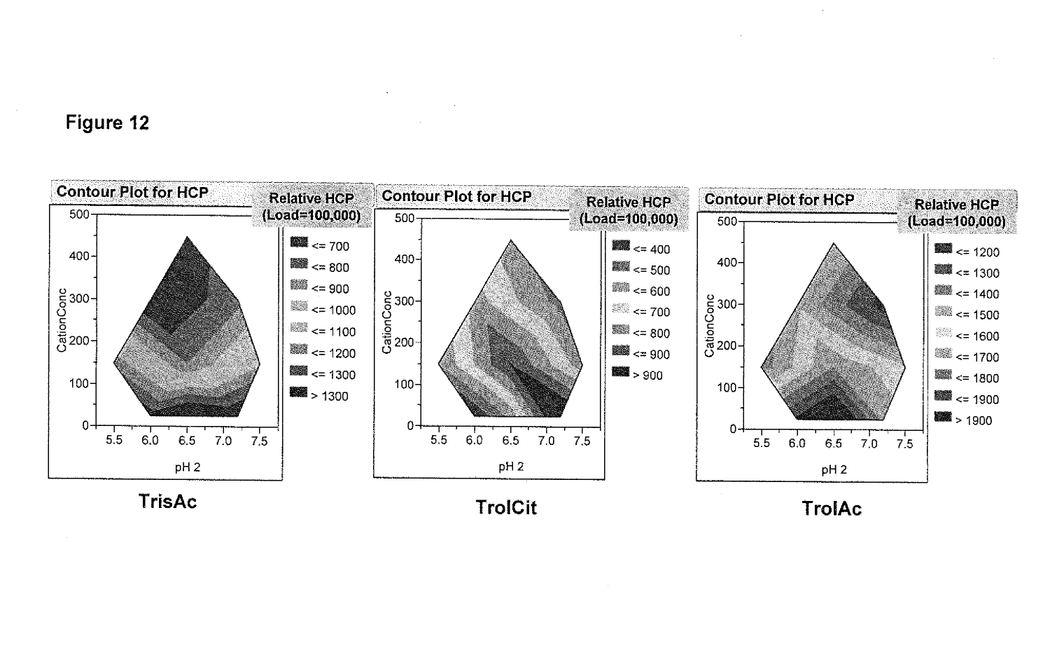

From the product quality results of the baseline runs, we were able to narrow down the list of suitable buffer systems by removing those containing phosphate or sodium ions, leaving only the Tris/trolamine acetate and trolamine citrate systems. For these buffer systems, the effect of Wash II cation concentration and buffer pH on Protein A performance in terms of yield aggregate and host cell protein (HCP) clearance were evaluated using a matrix as shown in FIG. 12.

The comparison runs were performed to evaluate Mabselect Sure capture performance using the simplified buffer systems for 2 other antibodies, Antibody B (also referred herein as “Molecule B”) and Antibody C (also referred herein as “Molecule C”). The wash buffers used were optimized from the results of the Antibody A (also referred herein as “Molecule A”) runs, and are shown in Table 5.

TABLE 5
Buffers Used for Comparison Runs

| System | Equilibration | Wash | Elution |
|---|---|---|---|
| Control | 25 mM Tris + | 20 mM NaCitrate, | 100 mM |
| | 100 mM NaCl | 500 mM NaCl | NaAcetate |
| | pH 7.2 | pH 6.0 | pH 3.5 |
| Tris | 25 mM Tris- | 300 mM Tris- | 25 mM Acetate |
| Acetate | Acetate | Acetate | (0.89 mM Tris) |
| | pH 7.2 | pH 7.2 | pH 3.5 |
| Trolamine | 25 mM Trol- | 300 mM Trol- | 25 mM Acetate |
| Acetate | Acetate | Acetate | (0.89 mM Trol) |
| | pH 7.2 | pH 7.2 | pH 3.5 |
| Trolamine | 25 mM Trol- | 300 mM Trol- | 25 mM Citric |
| Citrate | Citrate | Citrate | (Trol) Acid |
| | pH 7.2 | pH 7.2 | pH 3.5 |

Antibody concentrations in eluate samples obtained from the lab studies were determined by spectrophotometric analysis at UV280 nm. The method utilized duplicate 25-fold dilutions of test samples in 1× phosphate buffered saline (PBS), and were read on a Agilent 8453 UV/Visible Spectrophotometer (Agilent, Catalog #G1815AA, Santa Clara, Calif.) at a wavelength of 280 nm.

Antibody concentrations in clarified harvest material from the lab studies were determined by Poros A HPLC method. The method utilized duplicate 100 μL injections of 5 standards (0.025, 0.05, 0.10, 0.50, 1.0 μg/μL) for the standard curve and sample dilutions were applied to achieve readings within the standard curve range. A Shimadzu HPLC system was configured with a Poros A ImmunoDetection sensor cartridge (Applied Biosystems, Foster City, Calif.). The column was maintained at 20-22° C. and autosampler tray temperature was set at 4° C. The system was run at 2 mL/min for a run time of 10 minutes, absorbance was monitored at UV280 nm.

Aggregate levels in the eluate samples were determined by the SEC method. The method utilized five 20 μL injections of a reference standard at 1.0 μg/μL concentration and duplicate runs were done for each of the samples which were all diluted to an antibody concentration of 1.0 μg/μL. A Shimadzu HPLC system was configured with a TOSOH BioScience TSKgel G3000SWXL SEC column. The column was maintained at 20-22° C. and autosampler tray temperature was set at 4° C. The system was run at 0.50 mL/min for a run time of 60 minutes, absorbance was monitored at 214 nm and the buffer used was 100 mM sodium phosphate, 200 mM Sodium Sulfate pH 6.8.

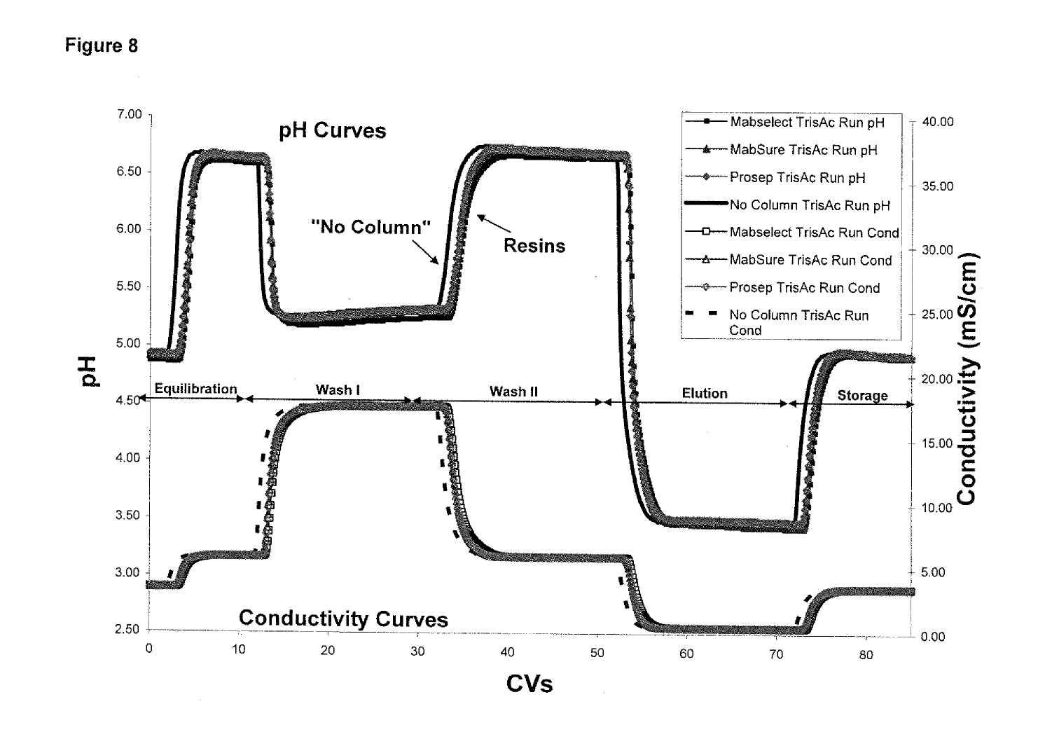

FIG. 8 shows an example of the chromatogram overlays generated from the first set of blank runs. FIG. 8 shows all the pH and conductivity transition curves in terms of CVs for the Tris acetate system for all three resins and the no column condition. The conductivity transition curves are at the bottom of the graph, while the pH transitions are at the top of the graph. The run that was performed with no column inline (shown as the line with no symbols) shows transitions that occur faster than with resins by ˜1 CV due to the absence of a column. Besides this difference, there are no other significant differences between the shapes of the transition curves for both pH and conductivity for all resin conditions. The same is observed for all the other acetate systems and citrate systems.

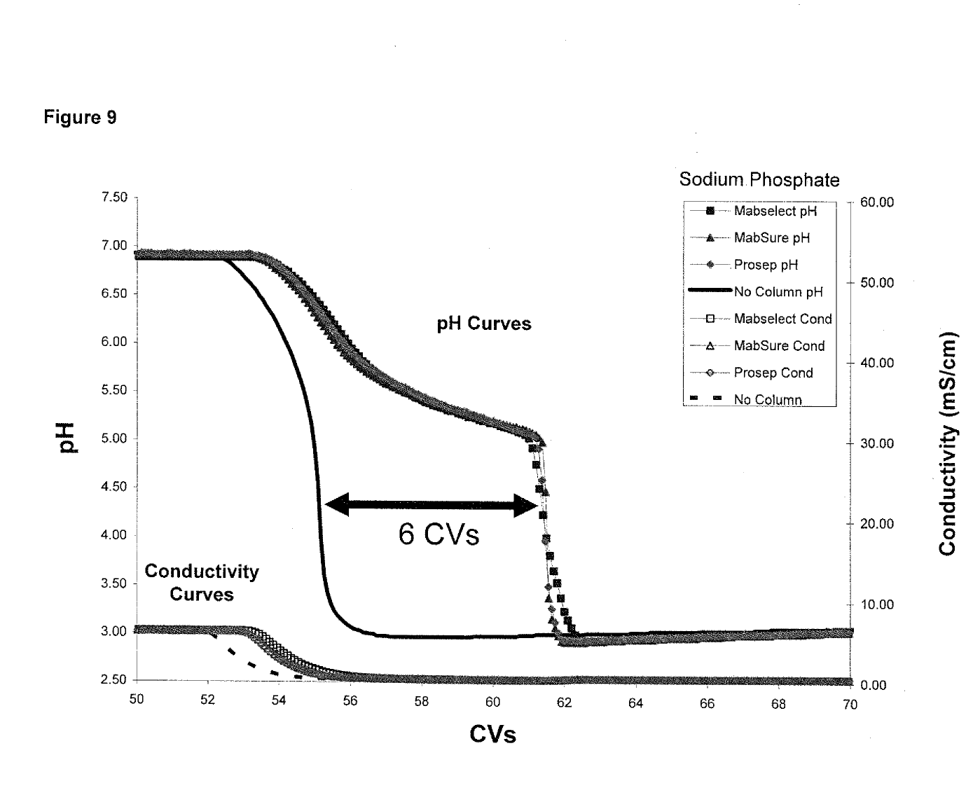

For the phosphate buffer systems, although the transitions look similar across the different resins, the pH transition for the elution curves are different from the no column condition, as can be seen in FIG. 9. There is a difference in pH curve shape, and there is a significant lag of about 6 CVs between the transition curves of the no column condition and the other 3 resins. This is due to the phosphate buffer interaction with the resin.

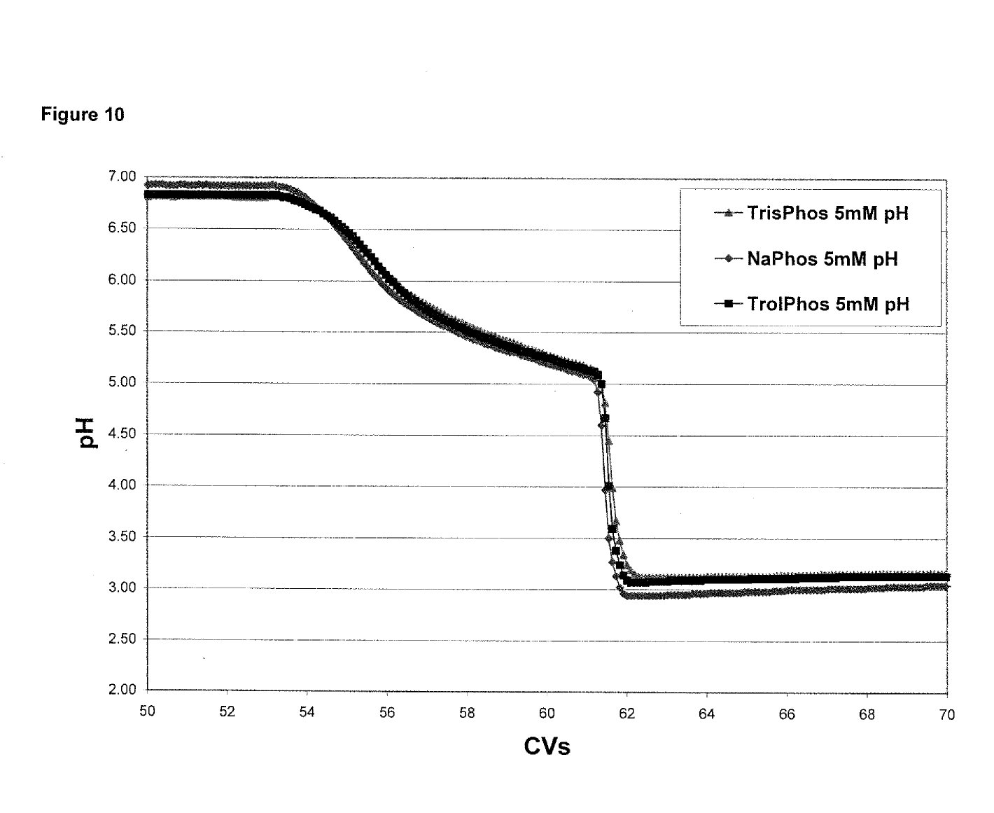

Comparing across different cations for the same anionic system and the same resin, it can be seen from FIG. 10 that the transitions in pH and conductivity are all similar, indicating that cationic components do not play a significant role in affecting pH transitions across Protein A resins.

Comparing across different anions for the same cationic system and the same resin, it can be seen from FIG. 114 that the anion significantly affects the shape of the pH transition curve, especially during the elution step.

Overall, it can also be seen that conductivity transitions are not affected by the resin used or the ionic components present. With the results obtained, it was decided to reduce the buffers considered to the Tris-containing buffer systems since the cationic effect on pH and conductivity transitions is not significant.

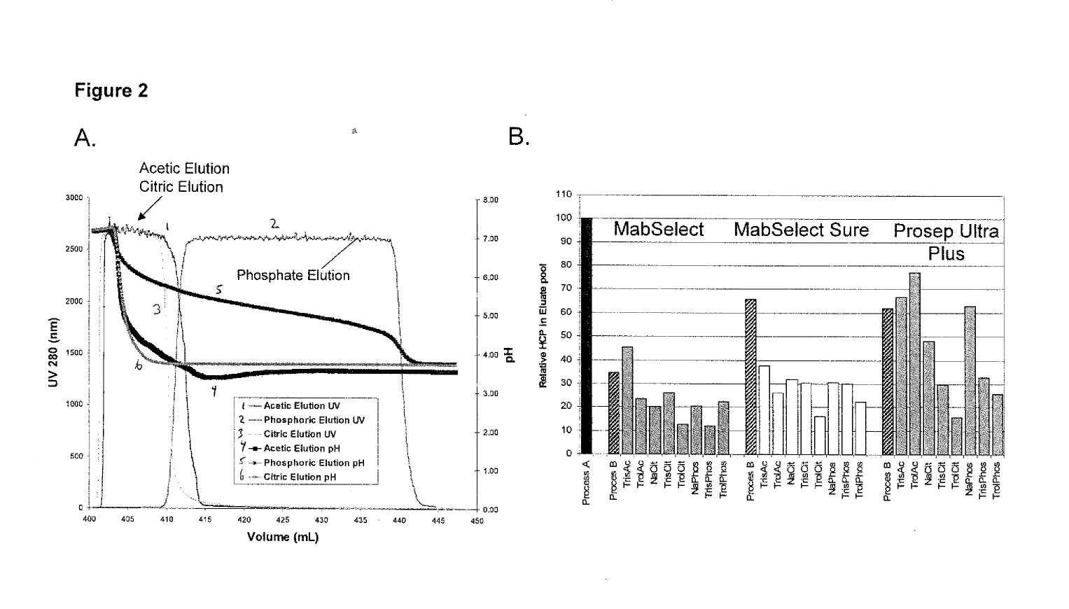

The product quality results in terms of HCP reduction for the different buffer systems can be seen in FIG. 2B. From FIG. 2B, it can be observed that in general, the Mabselect resins perform better than PUP in terms of HCP clearance for the same buffer system. It is also clear that certain buffer systems clear HCP better than others, like the citrate systems as compared to the acetate systems, and the Tris and Trolamine.

FIG. 12 shows the resultant contour plots obtained for each three buffer systems for Antibody A eluate HCP content as a function of different Wash 1I pH and cation concentrations. From the results obtained, it is clear that with higher wash II cationic concentration there is better impurity clearance, but the yield is slightly diminished (data not shown), pH does not seem to have a significant effect on HCP clearance for the ranges studied. The Trolamine citrate buffer system performs the best, having the lowest eluate HCP range of 400-900 ng/mg for the conditions studied. Hence, in certain embodiments, the conditions to be used for the Wash II phase for can be a cationic concentration of 300 mM and pH 7.2, and these were the conditions used for the Antibody B and Antibody C comparison runs.

Table 7 compares the eluate HCP content across different antibodies and different buffer systems. While HCP clearance performance is different between different antibodies, the simplified buffer systems seem to perform comparably or better than the conventional process buffers.

TABLE 7
Antibodies A/B/C Comparison of HCP Results*

| Antibody (ng/mg) | C | B | A |
|---|---|---|---|
| Conventional | 2580 | 589 | 1625 |
| Process | | | |
| Tris | 2508 | 1409 | 739 |
| Acetate | | | |
| Trolamine | 2604 | 1542 | 1103 |
| Acetate | | | |
| Trolamine | 2293 | 441 | 565 |
| Citrate | | | |

*relative to load = 100,000

From the blank runs, it was determined that all three Protein A resins behave similarly when subjected to the same buffer system, and cationic species do not have a significant effect on pH or conductivity transitions on the resins. Anionic species, however, affect the shapes of the transitions due to their pKa properties. It was also determined that the simplified buffer systems perform better at column equilibration as compared to conventional process buffers, and buffer concentrations are inversely proportional to equilibration volumes.

From the baseline runs, it was determined that different buffer systems perform differently with respect to HCP clearance, and Mabselect™ resins perform better than Prosep Ultra Plus™ in that respect. It was also determined that due to the gradual pH transition during elution for the phosphate buffer systems, it resulted in delayed and much larger eluate volumes.

From the results of the various runs, the intermediate wash conditions were optimized for product quality and yield performance to a cationic concentration of 300 mM and pH of 7.2.

Finally, the runs performed on different antibodies demonstrated that for the tris acetate, trolamine acetate and trolamine citrate systems, Protein A capture performance was comparable or better than when using conventional process buffers, indicating that these three buffer systems are suitable for a streamlined purification process using a single buffer system

### 2. Fine Purification

### 2.1 Anion Exchange Resin and Q Membrane Performance in Two-Component Salt-Free Buffer Systems

As a mAb purification platform fine purification step, the performance of anion exchange (AEX) media can be impacted by pH, conductivity, and buffer system components. Therefore, it is important to understand the performance of various anion exchange media, both resin and membrane, with different monoclonal antibody molecules under a range of conditions.

The objective of this study was to determine flow through mode AEX chromatography conditions by performing high-throughput screening (HTS) studies on various resins for two monoclonal antibodies. The effects of conductivity, pH, and protein concentration were evaluated in terms of product binding and impurity removal. At optimal conditions, impurity breakthrough curves were assessed for various AEX media.

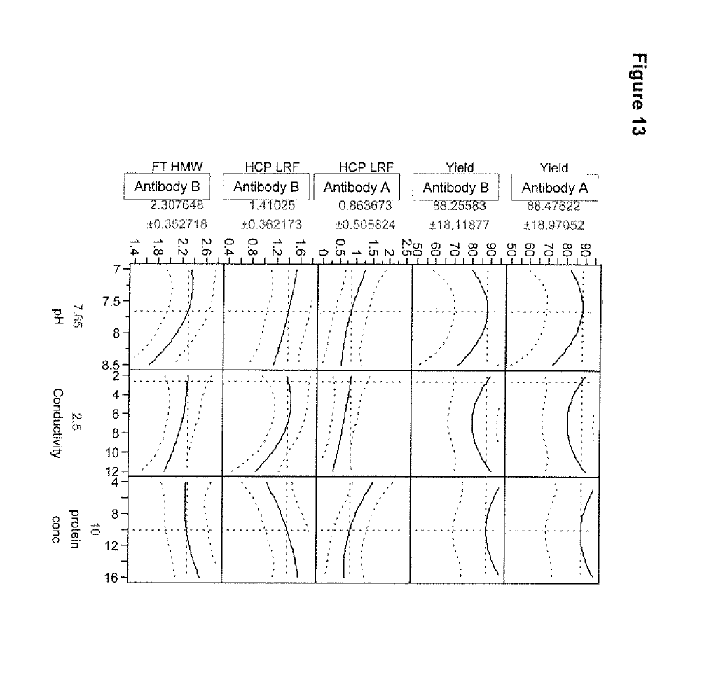

A study was performed to determine AEX operational ranges applying high throughput screening (HTS) in flow-through mode. The HTS was carried out using AEX resin in pre-loaded PreDictor plates (GE Healthcare). The load materials were prepared using both Antibody A and Antibody B molecules from MabSelect eluates in a two-component buffer system, Tris-Acetate. The variables were set up as pH from 7.0 to 8.5, conductivity from 2 to 12 mS/cm, and mAb concentration from 4 to 16 g/L. The load amount was 50 g of mAb per L of medium. Antibody A MabSelect eluate was adjusted to pH 3.5 with 3M Acetic acid for viral inactivation, then neutralized to designed pH using 3M Tris. The conductivity was controlled with the concentration of Tris and Acetic acid. The prepared load materials were centrifuged and filtered through 0.2 μm filter prior to loading. The results of this experiment are presented in FIG. 13.

With P value <0.05, the parameter impact is considered as significant. For example, the pH impacted product yield for both Antibody A and Antibody B. The highest product recovery yield was at pH 7.65. Furthermore, HCP log reduction factor was strongly affected by conductivity for both Antibody A and Antibody B. The interaction of conductivity and protein concentration, and the interaction of conductivity and pH had a significant impact on HCP reduction for Antibody A. A higher HCP LRF for both Antibody A and Antibody B was achieved with lower conductivity. pH significantly affected Antibody B aggregate percent. Higher pH resulted in lower percent of aggregates. Non-limiting conditions identified as effective: pH 7.7±0.1, and conductivity of 2.5±0.5 mS/cm.

With the non-limiting example of effective conditions (pH 7.7 and conductivity of 2.5 mS/cm, Tris-acetate buffer), four AEX resins were selected for further column chromatography study. Resin selection was based on preliminary HTS results of eight AEX resins. 1×10 cm columns were packed with each resin. Impurity breakthrough curves were obtained from the four AEX columns and two Q membranes for both Antibody A and Antibody B molecules. The Antibody A and Antibody B MabSelect eluates were viral inactivated at pH 3.5 with 3 M acetic acid, and neutralized to pH 7.7 with 3 M Tris. Conductivity was adjusted to 2.5 mS/cm by adding Milli Q water to the pH adjusted materials. These materials were centrifuged and filtered with 0.2 μm filter prior to loading.

For these experiments, the following conditions were employed: pH 7.7, 2.5 mS/cm, 47 mM Acetate/69 mM Tris; the following AEX Resins were employed: Q Sepharose FF (GE Healthcare); Toyopearl QAE 550C (Tosoh); Poros 50HQ (Applied Biosystems); and Poros 50PI (Applied Biosystems); and the following Q Membranes were employed: Sartobind (Sartorius) and ChromaSorb (Millipore).

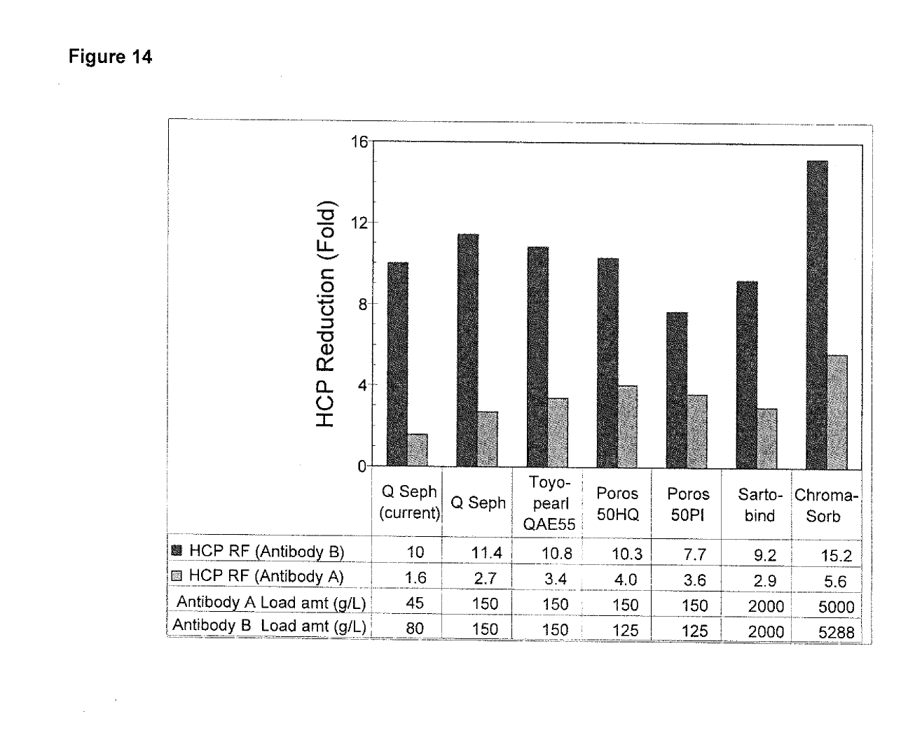

The results of these experiments are depicted in FIG. 14. In particular, four AEX resins had similar HCP removal capacity for each mAb: 2.7 to 4 fold HCP reduction for Antibody A and 8 to 11 fold HCP reduction for Antibody B. ChromaSorb Q had highest HCP removal capacity with 5000 g/L load: 5.6 fold HCP reduction for Antibody A and 15.6 fold HCP reduction for Antibody B.

In the next set of experiments, the conductivity and pH conditions were modified from the determined optimization of pH 7.7 and 2.5 mS/cm to be streamlined with the a Capto-Adhere™ column, which performs best at pH 7.9 and 4.5 mS/cm. In an effort to remove chloride from the Platform buffers, the simplified two-component buffer systems were designed to be salt-free. These buffers, which contained an anion component of either Acetate or Citrate, and a cation component of either Tris or Trolamine, were evaluated with Q membrane performance. Each of the four buffer systems was tested with one of the three Q membranes for a total of twelve runs. MabSelect eluate of Antibody A with the four buffer systems were viral inactivated at pH 3.5 by adding 3M Citric acid or 3M Acetic acid, then neutralized to pH 7.9 by adding 3M Tris or 3M Trolamine. The final conductivities were adjusted to 4.5 mS/cm with water. The materials were centrifuged followed with 0.2 μm filtration prior to membrane loading.

For these experiments the following load conditions were employed: pH: 7.9+0.1, Conductivity: 4.5+0.5 mS/cm, Load to 7,000 g/L, and wash with equilibration buffer until return to baseline; the following Buffer Systems were employed: Tris-Citrate; Trolamine-Citrate; Tris-Acetate; and Trolamine-Acetate; and the following Q Membranes were employed: Mustang Q; ChromaSorb Q; and Sartobind Q.

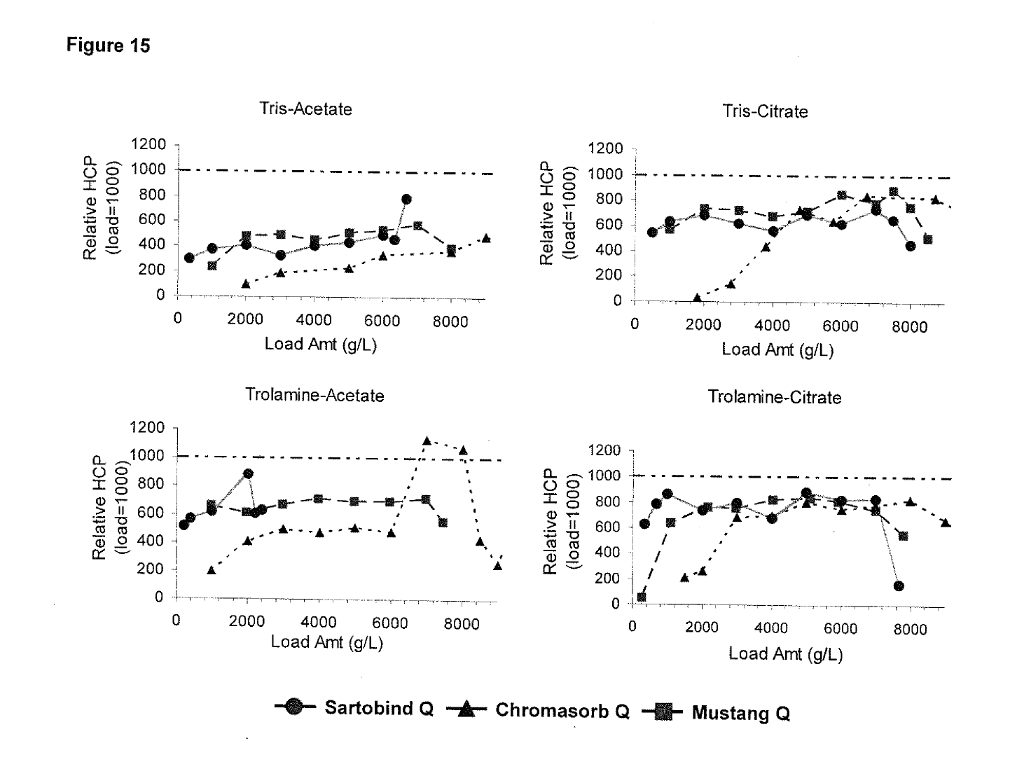

The results for these experiments are illustrated in FIG. 15. HCP breakthrough was the smallest for the ChromaSorb Q membrane compared to Sartobind Q and Mustang Q in the Tris-Acetate and Trolamine-Acetate buffer systems. In the Citrate buffer systems (Tris-Citrate and Trolamine-Citrate), ChromaSorb Q had significantly lower HCP breakthrough up to 3000 g/L load than Sartobind Q and Mustang Q. The Tris-Acetate buffer system had the least HCP breakthrough for all three membranes, especially ChromaSorb.

In summary, these experiments indicate the following: that HCP removal was strongly affected by conductivity. Lower conductivity resulted in higher HCP reduction. That aggregate level was significantly affected by pH. Higher pH resulted in lower aggregate percentage. That product recovery is affected by pH. Higher pH resulted in higher yield.

Overall HCP reduction was higher in the Tris-Acetate system compared to the other buffers. ChromaSorb in the Tris-Acetate system cleared leached Protein A up to 5000 g/L load, whereas the other membranes and systems showed no significant clearance aggregate profile. No significant aggregate increase or decrease observed in any membrane in any system. Reasonable recovery yield (>90%) for most buffers and systems with the exception of Sartobind in Trolamine Acetate (84%), which may have been due to insufficient load (2430 g/L load).

In addition, the results indicate a number of effects of the selected buffer systems. Tris-Acetate resulted in the best impurity clearance for all three Q membranes. The Tris-Acetate buffer also showed best performance for CaptoAdhere chromatography. Comparison of AEX media at the same conditions indicated that strong AEX resins (Q Sepharose, Toyopearl QAE, Poros 50HQ) were comparable in loading capacity and impurity removal. Q membranes had a higher loading capacity than strong AEX resins, particularly ChromaSorb Q. ChromaSorb load amount can potentially be increased according to the impurity concentration in the load material.

With regard to the performance of molecules loaded, the results indicated the following. Higher HCP reduction was observed for Antibody B than for Antibody A in all tested AEX resins and Q membranes. This may be due to the difference in HCP species and/or assay sensitivity.

### 2.2 Development of a MAb Platform Fine Purification Step Using Capto-Adhere™ Column Chromatography and a Streamlined Salt-Free Buffer System

Capto-Adhere™ is a mixed mode resin and is designed for post-protein A purification of monoclonal antibodies. It can remove contaminants such as leached protein A, HCP, DNA and aggregates. Its base matrix is a highly cross-linked agarose with a ligand (N-Benzyl-N-methyl ethanol amine) that exhibits many functionalities for interaction, such as ionic interaction, hydrogen bonding and hydrophobic interaction. It is manufactured by GE Healthcare.

Capto-Adhere™ was evaluated as a single fine purification step, in flow through mode, in a two-column MAb (Antibody A and Antibody B) purification platform process. Studies targeting high loading capacity were applied to define operational conditions using a simplified two-component salt-free buffer system. Four buffer systems were compared and the Tris-Acetate buffer system performed well with regard to impurity reduction.

In the initial set of experiments in this study, the following conditions were employed to evaluate buffer conditions: Resin column: 4.7 mL HiScreen Capto-Adhere™ (GE Healthcare); Tris-Acetate buffer; pH: 7.0-8.2; Conductivity: 4-12 mS/cm; Load: Antibody A up to 300 g/L resin; Antibody B up to 250 g/L resin; pH was adjusted using 3M Tris or 3M Acetic acid, conductivity was controlled with the concentration of Tris-acetate.

Antibody recovery yield was significantly affected by both pH and conductivity; Antibody B recovery yield was only significantly affected by the interaction of conductivity. Lower conductivity and lower pH results in higher yields for both antibodies. Leached protein A levels were significantly affected by conductivity for both antibodies; pH had minor impact on protein A levels for Antibody B. Lower conductivity results in lower leached protein A concentration in flow through for both antibodies. HCP reduction was significantly affected by conductivity for both antibodies. HCP removal was significantly affected by pH, and the interaction of pH & conductivity for Antibody A. pH had a minor effect on HCP removal for Antibody B.

Lower conductivity and higher pH results in higher HCP reduction for Antibody A. Lower conductivity results in higher HCP reduction for Antibody B. Product monomer % was significantly affected by conductivity and pH for both antibodies. Lower conductivity and higher pH results in higher product monomer %. Total yield for both antibodies was >91.4% for all conditions.

In light of the foregoing, non-limiting effective conditions for acceptable yield and product quality include: Conductivity: 4.0-5.0 mS/cm; pH: 7.8-8.0; and Column loading: 150-200 g/L resin. Comparable results were obtained with two other mixed mode resin, HEA-HyperCel (hexylamine) and PPA-HyperCel (propylphenyl amine) (Pall).

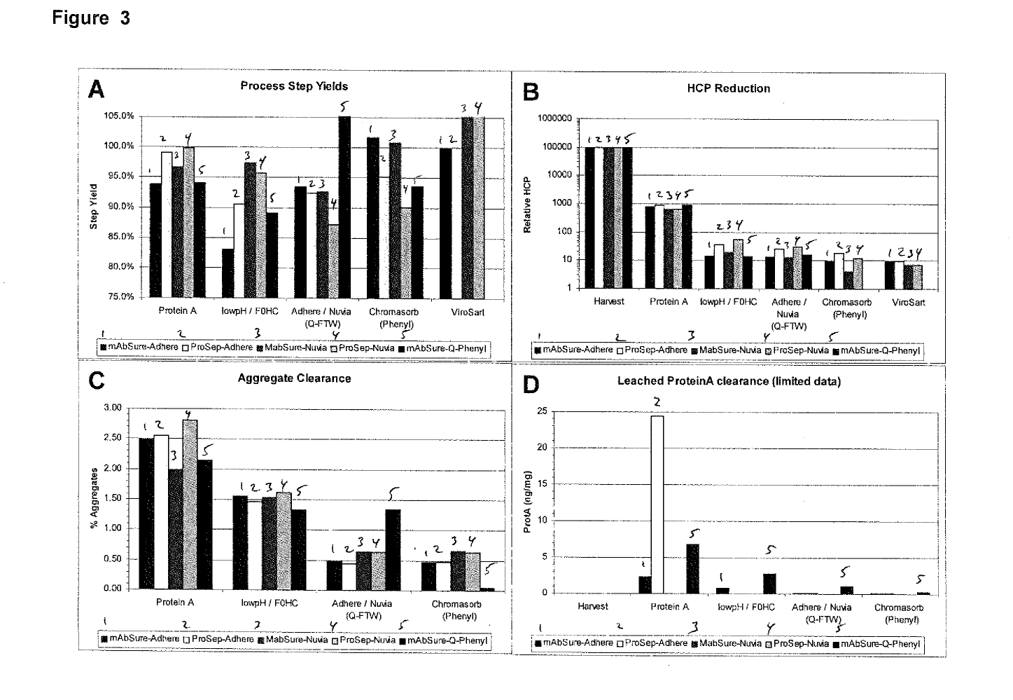

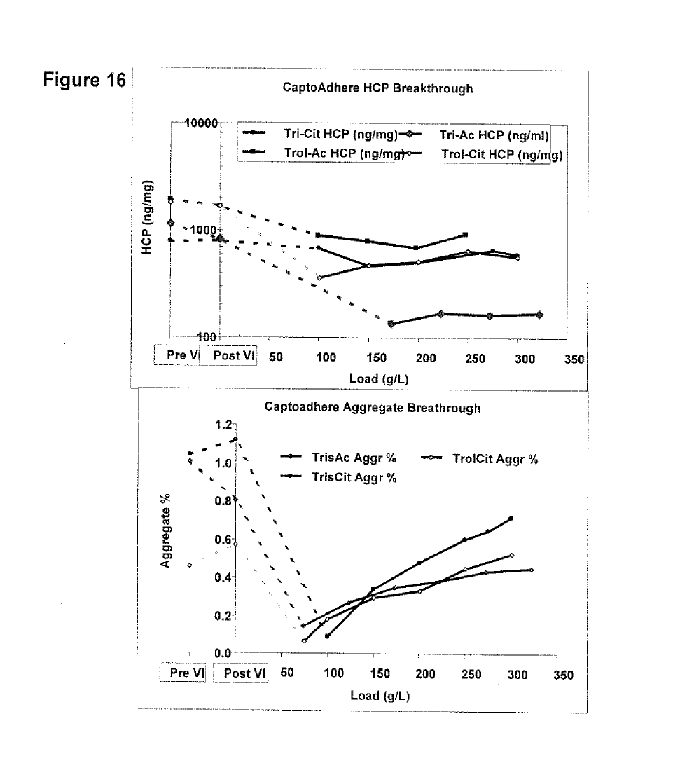

Simplified two-component buffer systems, at optimized conditions, were further evaluated for their impact on impurity reduction using the following conditions: buffer systems: Tris-Citrate; Trolamine-Citrate; Tris-Acetate; Trolamine-Acetate; pH: 7.9±0.1; Conductivity: 4.5±0.5 mS/cm; Load: 300 g/L resin. As illustrated in FIG. 16, viral inactivation of load resulted in an increase in aggregation for Tris-Citrate and Trolamine-Citrate buffer systems, but not Tris-Acetate. Viral inactivation of load material resulted in a decrease in HCP in Tris-Acetate, Trolamine-Acetate, and Trolamine-Citrate buffer systems. Tris-Acetate showed the greatest HCP reduction at all loadings tested. Tris-Acetate showed the least aggregate breakthrough at the high loading amount. Tris-Citrate buffer showed faster aggregate breakthrough. Tris-Acetate and Trolamine-Citrate showed similar aggregate breakthrough in the low loading range (Trolamine-Acetate buffer data not shown due to significant aggregation). Based on the information from the experiments described herein, integrated platform processes, from clarification to purification, were executed at bench-scale using Tris-Acetate salt-free buffer system throughout the entire process. The integrated purification process steps for Antibody A (as shown below) are compared in FIG. 3 and one integrated process was also demonstrated for Antibody B.

Antibody A:
MabSelect SuRe—F0HC—CaptoAdhere—ChromaSorb Q—ViroSart;
Prosep Ultra Plus—F0HC—CaptoAdhere—ChromaSorb Q—ViroSart;
MabSelect SuRe—F0HC—Nuvia S—ChromaSorb Q—ViroSart;
Prosep Ultra Plus—F0HC—Nuvia S—ChromaSorb Q—ViroSart; and
MabSelect SuRe—F0HC—Q Sepharose—Phenyl HP Sepharose.

Antibody B:
MabSelect SuRe—F0HC—CaptoAdhere—ChromaSorb Q—ViroSart

All platform processes evaluated produced comparable viral filtrate. Antibody A HCP was much lower than the existing processes. Capto-Adhere™, as a flow through mode, improved process throughput and process time, although β-glucan could not be reduced by Capto-Adhere™.

In light of the foregoing, Capto-Adhere™ chromatography, developed as a fine purification step, was demonstrated in a MAb purification platform process using a streamlined two-component salt-free buffer system. Non-limiting effective operational ranges for Capto-Adhere™were determined to be: Buffer: Tris-acetate buffer (90 mM Acetate/˜125 mM Tris) with pH 7.9±0.1 and conductivity of 4.5±0.5 mS/cm; Loading range: 150-200 g/L

In addition, it was identified that Antibody A HCP is significantly lower than the conventional process and that Antibody B HCP is comparable to conventional process With flow through mode and high loading capacity, Capto-Adhere™ has a significantly reduced process time compared to the conventional processes.

Various publications are cited herein, the contents of which are hereby incorporated by reference in their entireties.

What is claimed is:

1. A method for producing a host cell protein-reduced antibody preparation from a sample mixture comprising an antibody and at least one host cell protein, said method comprising:
(a) contacting said sample mixture with a loading buffer and contacting said loading buffer and sample mixture to a capture separation chromatographic support under conditions where said antibody is retained on said chromatographic support;
(b) washing said capture separation chromatographic support with a wash buffer to remove the sample mixture components that are not retained on said capture separation chromatographic support; and
(c) contacting said capture separation chromatographic support with an elution buffer to thereby produce a capture separation eluate;
wherein said loading, wash, and elution buffers consist of water and essentially the same anion and cation components; and
wherein said capture separation eluate comprises said host cell protein-reduced antibody preparation.

2. The method of claim 1, wherein said anion and cation components are selected from the group consisting of Tris and Citrate, Tris and Acetate, Trolamine and Citrate, and Trolamine and Acetate.

3. The method of claim 1, wherein said capture separation chromatography support is a Protein A resin selected from the group consisting of a MabSelect™ resin (GE Healthcare), MabSelect Sure™ resin (GE Healthcare), and ProSep Ultra Plus (Millipore).

4. The method of claim 1, comprising the steps of:
(d) contacting said capture separation eluate to a loading buffer and contacting said capture separation eluate and loading buffer mixture to a fine purification separation chromatographic support capable of further reducing the host cell protein content of the capture separation eluate; and
(e) washing said fine purification chromatographic support with a wash buffer to remove the capture separation eluate components that are not retained on said fine purification chromatographic support; and
(f) contacting said fine purification chromatographic support with an elution buffer to thereby produce a fine purification separation eluate
wherein the capture separation and fine purification separation load, wash, and elution buffers consist of water and essentially the same anion and cation components selected from the group consisting of Tris and Citrate, Tris and Acetate; Trolamine and Citrate; and Trolamine and Acetate

5. The method of claim 4 wherein said fine purification separation chromatographic support is an ion exchange matrix selected from the group consisting of an anion exchange matrix and a cation exchange resin; a mixed mode resin or a hydrophobic interaction resin.

6. The method of claim 5, wherein said ion exchange matrix is a cation exchange resin selected from the group consisting of Fractogel, carboxymethyl (CM), sulfoethyl(SE), sulfopropyl(SP), phosphate(P), sulfonate(S), Nuvia S (BioRad), Capto S (GE Healthcare) and Gigacap S (Tosoh); or an anion exchange matrix selected from the group consisting of Q sepharose, diethylaminoethyl (DEAE), quaternary aminoethyl(QAE), quaternary amine(Q) groups, Q Sepharose FF (GE Healthcare), Toyopearl QAE 550C (Tosoh), Poros 50HQ (Applied Biosystems), Poros 50PI (Applied Biosystems), Sartobind Q (Sartorius), ChromaSorb Q (Millipore) and Mustang Q (Pall).

7. The method of claim 5, wherein said chromatographic support is a mixed mode resin selected from the group consisting of Capto-Adhere™ (GE Healthcare), HEA-HyperCel (hexylamine) and PPA-HyperCel (propylphenyl amine) (Pall); a hydrophobic interaction resin selected from the group consisting of alkyl-, aryl-groups, Phenyl Sepharose, Phenyl Sepharose™ 6 Fast Flow column, Phenyl Sepharose™ High Performance column, Octyl Sepharose™ High Performance column, Fractogel™ EMD Propyl, Fractogel™ EMD Phenyl columns, Macro-Prep™ Methyl, Macro-Prep™ t-Butyl Supports, WP HI-Propyl (C₃)™ column, Toyopearl™ ether, phenyl or butyl columns and a combination thereof.

8. The method of claim 1 further comprising a depth filtration step, a viral inactivation step selected from the group consisting of a viral filtration step and a pH-mediated viral inactivation step, or a combination thereof.

9. A method for developing an integrated purification protocol for producing host cell-reduced preparations from two sample mixtures where each sample mixture comprises a distinct antibody and at least one host cell protein:
(a) selecting a capture separation chromatographic support capable of retaining the distinct antibodies of said sample mixtures; and
(b) selecting load, wash, and elution buffers for, respectively, loading, washing, and production of a capture separation eluate;
wherein said loading, wash, and elution buffers consist of water and essentially the same anion and cation components; and
wherein said capture separation eluate comprises said host cell protein-reduced antibody preparation.

10. The method of claim 9, wherein said anion and cation components are selected from the group consisting of Tris and Citrate, Tris and Acetate, Trolamine and Citrate, and Trolamine and Acetate.

11. The method of claim 9, wherein said capture separation chromatography support is a Protein A resin selected from the group consisting of MabSelect™ resin (GE Healthcare), MabSelect Sure™ resin (GE Healthcare), and ProSep Ultra Plus (Millipore).

12. The method of claim 11, further comprising the steps of:
(c) selecting a fine purification separation chromatographic support capable of further reducing the host cell protein content of the capture separation eluate; and
(d) selecting load, wash, and elution buffers for, respectively, loading, washing, and production of a fine purification separation eluate;
wherein the capture separation and fine purification separation buffers consist of water and essentially the same anion and cation components selected from the group consisting of Tris and Citrate, Tris and Acetate, Trolamine and Citrate, and Trolamine and Acetate.

13. The method of claim 12 wherein said fine purification separation chromatographic support is an ion exchange matrix selected from the group consisting of an anion exchange matrix or a cation exchange resin.

14. The method of claim 13, wherein said ion exchange resin is a cation exchange resin selected from the group consisting of Fractogel, carboxymethyl (CM), sulfoethyl(SE), sulfopropyl(SP), phosphate(P), sulfonate(S), Nuvia S (BioRad), Capto S (GE Healthcare) and Gigacap S (Tosoh); or an anion exchange matrix selected from the group consisting of Q sepharose, diethylaminoethyl (DEAE), quaternary aminoethyl(QAE), quaternary amine(Q) groups, Q Sepharose FF (GE Healthcare), Toyopearl QAE 550C (Tosoh), Poros 50HQ (Applied Biosystems), Poros 50PI (Applied Biosystems), Sartobind Q (Sartorius), ChromaSorb Q (Millipore) and Mustang Q (Pall).

15. The method of claim 12, wherein said fine purification separation chromatographic support is a mixed mode resin or a hydrophobic interaction resin.

16. The method of claim 15, wherein said chromatographic support is a mixed mode resin selected from the group consisting of Capto-Adhere™ (GE Healthcare), HEA-HyperCel (hexylamine) and PPA-HyperCel (propylphenyl amine) (Pall); or a hydrophobic interaction resin selected from the group consisting of alkyl-, aryl-groups, phenyl sepharose, Phenyl Sepharose™ 6 Fast Flow column, Phenyl Sepharose™ High Performance column, Octyl Sepharose™ High Performance column, Fractogel™ EMD Propyl, Fractogel™ EMD Phenyl columns, Macro-Prep™ Methyl, Macro-Prep™ t-Butyl Supports, WP HI-Propyl (C₃)™ column, and Toyopearl™ ether, phenyl or butyl columns, and a combination thereof.

17. The method of claim 11 further comprising a depth filtration step, a viral inactivation step selected from the group consisting of a viral filtration step and a pH-mediated viral inactivation step, or a combination thereof.

18. A method for producing a host cell protein-reduced antibody preparation from a sample mixture comprising an antibody and at least one host cell protein, said method comprising:
(a) contacting said sample mixture with a loading buffer and contacting said loading buffer and sample mixture to a capture separation chromatographic support under conditions where said antibody is retained on said chromatographic support;
(b) washing said capture separation chromatographic support with a wash buffer to remove the sample mixture components that are not retained on said capture separation chromatographic support;
(b) contacting said capture separation chromatographic support with an elution buffer to thereby produce a capture separation eluate comprising said antibody;
(c) contacting said capture separation eluate to a second capture separation chromatographic support under conditions where said antibody is retained on said second capture separation chromatographic support, wherein said second capture separation chromatographic support is selected from the group consisting of an ion exchange matrix, a mixed mode resin and a hydrophobic interaction resin; and
(d) contacting said second capture separation chromatographic support with an elution buffer to thereby produce a second capture separation eluate,
wherein said loading, wash, and elution buffers consist of water and essentially the same anion and cation components; and
wherein said second capture separation eluate comprises said host cell protein-reduced antibody preparation.

19. The method of claim 18, wherein said anion and cation components are selected from the group consisting of Tris and Citrate, Tris and Acetate, Trolamine and Citrate, and Trolamine and Acetate.

20. The method of claim 18 further comprising a depth filtration step, a viral inactivation step selected from the group consisting of a viral filtration step and a pH-mediated viral inactivation step, or a combination thereof.
